(19) United States
(12) Patent Application Publication
(10) Pub. No.: US 2023/0008561 A1
(43) Pub. Date: Jan. 12, 2023

(54) Software Platform And Integrated Applications For Alcohol Use Disorder (AUD), Substance Use Disorder (SUD), And Other Related Disorders, Supporting Ongoing Recovery Emphasizing Relapse Detection, Prevention, and Intervention

(71) Applicant: Thomas Stevens, Chico, CA (US)

(72) Inventor: Thomas Stevens, Chico, CA (US)

(21) Appl. No.: 17/861,146

(22) Filed: Jul. 8, 2022

Related U.S. Application Data
(63) Continuation-in-part of application No. 16/945,803, filed on Aug. 1, 2020, now abandoned.
(60) Provisional application No. 63/041,112, filed on Jun. 18, 2020.

(51) Int. Cl. G16H 20/70; G16H 10/20; G16H 40/67; G16H 80/00; G06V 40/16

(52) U.S. Cl. CPC G16H 20/70; G16H 10/20; G16H 40/67; G16H 80/00; G06V 40/172

(57) ABSTRACT

A medical device combines a platform and an application on a user's mobile device to directly and continuously monitor the user's relapse condition based on sets of rules and AI analysis of a plurality of data points that are evaluated against a standard deviation (or other deviations) established from a history of the user and similarly situated individuals. Interaction and network support based or supplemented with knowledge of the user's emotional state and risk of relapse. The user's emotional state may be determined in a number of interrelated and/or independent ways including asking the user to rate his/her state, games/quizzes, and/or pictures of the user, which may be analyzed for emotional cues. The user may be engaged or user's network notified if conditions point to a likely or imminent relapse and a response team may be automatically selected and coordinated in a virtual and/or physical intervention.

### CROSS REFERENCE TO RELATED APPLICATIONS

This application is a Continuation-In-Part of U.S. patent application Ser. No. 16/965,803, filed 1 Aug. 2020, entitled “Method And Apparatus For Treatment And Relapse Prevention in Alcohol, Chemical, and other Dependencies,” which itself claims priority to U.S. Provisional Patent Application Ser. No. 63/041,112, filed on 18 Jun. 2020, entitled “Method And Apparatus For Treatment And Relapse Prevention in Drug, Alcohol, and other Dependencies,” the contents of each are hereby incorporated by reference in their entireties.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

### Field of Invention

The present invention relates to alcohol and chemical dependency related treatments, relapse prevention, ongoing recovery support.

### Description of Related Art

Various drug, alcohol, and other dependency treatments are known.

### SUMMARY OF THE INVENTION

The present inventor has realized the need for real-time evaluation of environment and circumstances for reminders, structured advice, warnings, and/or all-out intervention in dependency situations to facilitate treatment and to catch, stall, and respond before or within early stages of the relapse Process. The present invention comes in many embodiments and no single feature or component of one embodiment is exclusive thereto or required in any other embodiment even if described or implied as important to a particular embodiment.

In one embodiment, an application is provided that runs on an individual/patient's mobile device, tracking movement and monitoring activities which are applied to statistical data collected from any or all of the patient, the patient's peer group, Patients clinical care team, Social worker, patient's friends/family/social network (including on-line presences, posts, contacts, etc.), and national data/statistics of other patients similarly situated—for example. Decisions may be made based on, for example, the statistical averages, Artificial Intelligence (AI) (e.g., decision rules vetted over a larger database of drug and alcohol based dependencies), and such vetting performed using a machine learning (ML) API or other source.

The events and activities monitored include emotional cues and events/activities related to emotional cues such that, for example, allow for an accurate assessment of an emotional state or current emotional stability of the individual. Such assessment may be compared with statistical averages or compared against the individual's personal history and/or correlated against other events that may also be tracked.

In one embodiment, various decisions or processes may be run on the user's mobile device, on a back-end process hosted in the cloud, and/or split between local processing and cloud based servers or other computing means. The data may be analyzed to produce alerts/notifications as, for example, set-up in the application wherein the user may, for example, grant permission (e.g., opt-in permissions for monitoring, scanning, review, voice recognition, voice-to-text, key-word search, mobile device control such as camera and microphone, location data, and other data/analysis, applied statistics, analytics, etc., some or all of which may otherwise be private or confidential). The notification may be for example, notification or reminders to the user and may include pop-up photos or other information, suggested courses of action, etc. Such notification and/or reminders may be designed, for example, to prevent, forestall (or stall enough time for an intervention) actions by the user that have been determined to be statistically likely such as the purchase of alcohol or tobacco.

Data collection may include standard data collection devices commonly included in a mobile device e.g., location data, inertial data (e.g. IMU/accelerometer data collection), etc., and/or specialized medical or data collection equipment such as wearables, patches, printed, implantable devices, etc. The data collected includes all types of biometric or physical data related to the user such as blood pressure/heart rate (e.g., full EKG), galvanic skin response, glucose, alcohol air analysis.

The alcohol air analysis may be provided, for example, by a breathalyzer like device that does not need to be breathed into—detecting the smallest quantities possible of alcohol or alcohol laced vapors—not necessarily from the user (e.g., could be the surrounding environment—such as a nearby restaurant serving alcohol). A positive analysis would likely raise the possibility of user consumption and may signal an alert to a sober network leader, case manager, or Clinical Team, for example.

In one embodiment, the medical equipment may comprise, for example, a commercially available fitness device. For example, an API or other interface configured to access a Fitbit or similar device. Such API may be configured to directly access the device, or access a cloud storage/database populated using data from the device, for example. Regardless of the methodology, the data may be collected for analysis and correlation to events, diary entries, doctor/therapist notations, etc., and may be used to find patterns of activity as they relate to the patient and/or his/her treatment.

The various embodiments may include or be realized as, for example, a device, method, or Platform configured to track events and emotional status of an individual and correlate the events and emotional status against past events and emotional statuses to identify when overall circumstances for the individual have converged or are converging toward a likelihood of relapse. Notifying at least one of the individual, Sober Network, healthcare provider, therapist, or other stakeholders in the individual's recovery (friends, family, etc.) when such circumstances exist. Feedback to the user may be used, for example, to forestall the individual's behavior or change their course of action. Feedback to the individual may be used to help the individual recognize/acknowledge that current circumstances are not favorable and potentially allow them to recognize the danger and take some action to prevent full relapse.

Portions of the embodiments, whether a device, method, or other form, may be conveniently implemented in programming on a general purpose computer, or networked computers, and the results may be displayed on an output device connected to any of the general purpose, networked computers, or transmitted to a remote device for output or display. In addition, any components of any embodiment represented in one or more computer program or module(s), data sequence(s), and/or control signal(s) may be embodied as an electronic signal broadcast (or transmitted) at any frequency in any medium including, but not limited to, wireless broadcasts, and transmissions over copper wire(s), fiber optic cable(s), and co-ax cable(s), etc.

### BRIEF DESCRIPTION OF THE DRAWINGS

A more complete appreciation of the various embodiments and many of the attendant advantages thereof will be readily obtained as the same becomes better understood by reference to the following detailed description when considered in connection with the accompanying drawings, wherein:

FIG. 1A is a drawing of an architecture according to an embodiment;

FIG. 1B is a drawing of an architecture, communications links, and processes according to an embodiment;

FIG. 2 is an example relational database organized to keep track of recovery and other relevant data according to an embodiment; and

FIG. 3 is a flow chart of a data collection process according to an embodiment;

FIG. 4 is a flow chart of an analysis process according to an embodiment;

FIG. 5A is a drawing of biometric collection apparatus according to embodiments of the invention;

FIG. 5B is an illustration of collected biometrics data and an example of how the data may correspond and be utilized according to embodiments;

FIG. 6 is an illustration of a user interface according to an embodiment;

FIG. 7 is an illustration of a user interface and map according to an embodiment;

FIG. 8 is an illustrative flow of a scenario according to an embodiment;

FIG. 9 is a multi-dimensional graph illustrating the interplay of one combination of data-points according to an embodiment;

FIG. 10 is graph of a user's emotional state according to an embodiment; and

FIG. 11 is an output provided to a user to show the user emotional conditions over time according to an embodiment.

### DESCRIPTION OF THE PREFERRED EMBODIMENTS

Relapse is the single most difficult aspect to manage when facing Alcohol and Chemical dependency issues. One factor in success relates to catching the relapse upfront, preferably prior to a full relapse episode. Various embodiments described herein provide individuals who are struggling with Alcohol and Chemical dependency issues to become more aware in real-time, of hidden dangers that lead to relapse. Such awareness may be based on identified activities, actions, or circumstances from national averages or statistics and/or similar information specifically collected from the individual, or a combination of both.

In one embodiment (which like other embodiments may be a feature or features available from any embodiment) facilities are provided for reaching out and/or mandatory help, discussion or intervention (which may, for example, be court ordered, a rehab requirement, etc.). Such facilities are valuable because the inability to reach out for help, regardless of cause, has the potential to cripple even the most devoted sufferers in crisis and/or difficult circumstances. By collecting various personal, emotional, and situational data points throughout the recovery process, the application (app) (or backend in communication with the application) can track and help identify emotional, environmental, and social triggers that lead to relapse issues. Complex relationships to past known events and outcomes correlated to current circumstances are utilized to provide advance and ‘just in time’ warnings to individuals in imminent relapse danger.

Healthcare providers, emergency response team members, stakeholders, etc. may also be notified as needed and/or as guided by, for example, preferences (which may be mandatory, for example in some treatment programs, insurance reimbursement, etc.). In one embodiment, the individual/user/patient may have the ability or provided permission to alter preferences, which may, for example, be automatically populated based on a selected program. In another embodiment, the preferences are required for entry into a rehab or other treatment facility, for example. The mobile app may be “tied” to the individual via, for example, biometrics (e.g., periodic and/or continual data collection from one or more devices, wearables, implants, etc.) and alerts sent to providers if the associated signals are lost which is then alerted to the healthcare or other provider, for example.

Other data may include, for example, social media and analysis of posts, friends' posts (thumbs up, likes, comments etc.), seemingly unrelated data such as electricity use at home, activities at home identified by analysis of electrical signals on power lines, etc.

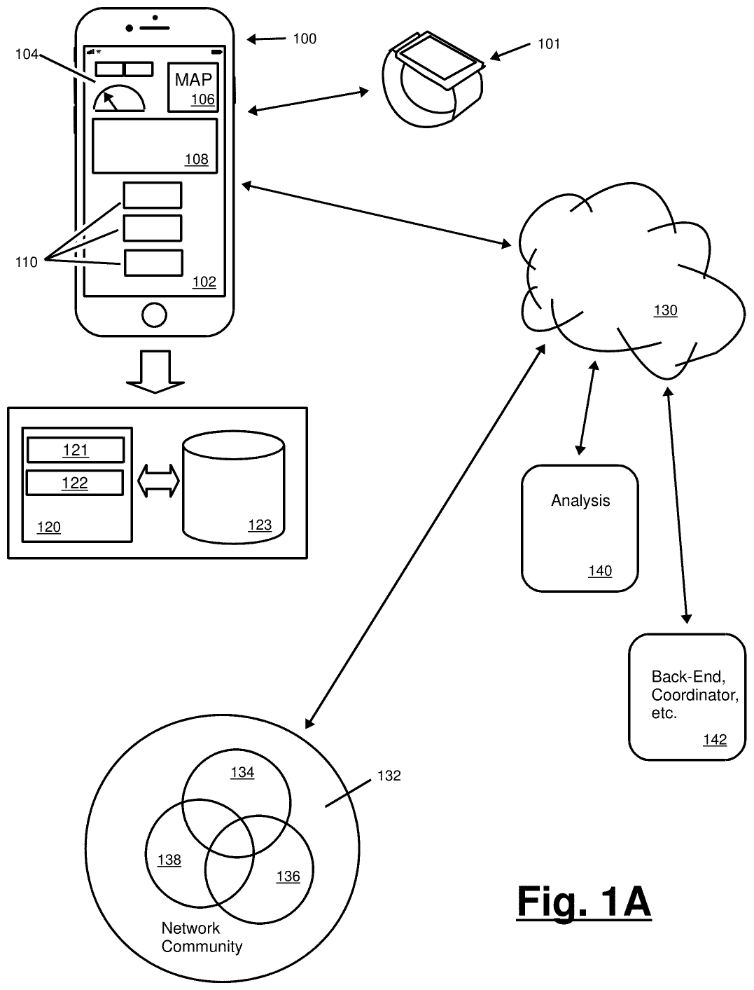

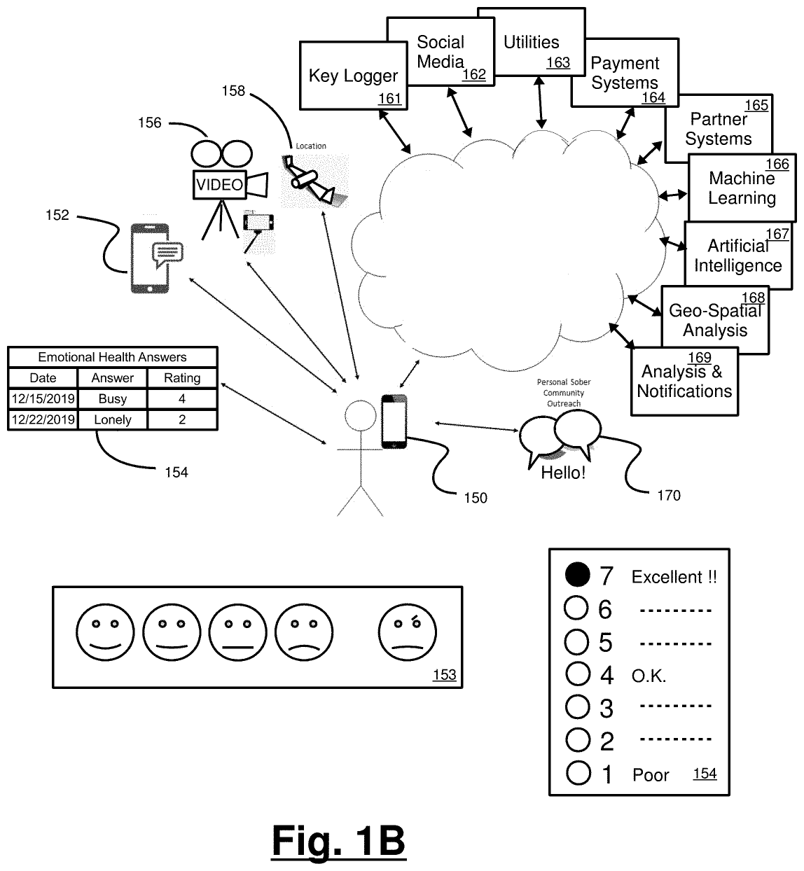

Referring now to the drawings, wherein like reference numerals designate identical or corresponding parts, and more particularly to FIG. 1 thereof, there is illustrated FIG. 1 is a drawing of an architecture including data points and processes according to an embodiment.

The architecture may include one or more features (again no single feature being a requirement), for example:

Mobile First

Designed for ease-of-use. Access your community anywhere on any device. For example, an application (app) on the user's mobile device or access through an Internet terminal, web browser, etc.

Community

The user build's his or her community to support sobriety.

Data-Driven

Based on the latest research, triggers and warnings are identified to keep the user on track (e.g., provide timely suggestions or warnings to the user, notification to stakeholders, etc.)

Customizable

The user may create or establish the settings and community that works best for the user.

Monitors

Keeps track of the user's triggers and environment to help the user get in front of a relapse.

Secure

The individual/user may manage and choose alerts and team members. The data is maintained secure and safe.

A user's mobile device 100 is illustrated with a user interface 102 (as provided by, for example app 121) with a number of optional features. Local processing power (processor 120) executes app 121, and other supporting functions 122 that may be utilized as needed for analysis, communication, or storage, and/or implement one or more features of the app/application. Data storage 123 may be utilized as needed to present the interface, retrieve (and/or process/pre-process) data such as, for example, biometric data from wearable device 101 (or other device, implant, or external equipment observing the user in some manner). Such data may be, for example, galvanic skin response, heart rate, blood pressure, blood oxygen content, electrical or neurological signals, etc. Such data may be pre-processed on device 100 or raw and communicated over a network (e.g., cloud 130) or other communication facility (channels in cloud 130, VPN, etc., for example) to specialized analysis module(s) (e.g., analysis 140) and/or backend 141, for example.

The various features may include, for example, status area 104 which may include an indication of status or boxes for points, scores, and/or dollars—any of which that may be utilized to ascertain completion of tasks, levels obtained (e.g., weeks of sobriety, points accumulated answering quizzes, relapses avoided, etc.) cumulative totals that may be increased or changed over time.

A MAP button 106 may provide fast access to locations of support individuals, standard or pre-planned routes or otherwise highlight other important features (e.g., scheduled locations or activity locations, etc.). A notification area 108 may provide an area for feeds or notifications (e.g., an alert or message from a counselor, community member, or other stakeholder). Buttons 110 may be programmed to initiate one or more features such as taking a quiz, providing a check-in, etc. Such buttons may be highlighted, illuminated, or flashed as a notification that action is needed (e.g., check-in past due).

Various, storage, processing, accounting, and communications may be performed via a network (e.g., cloud 130). Such communication may include, for example, communication with community members 132 which may be a combination of stakeholders including friends 134, medical 136, and/or on-call or reactionary forces 138. Advanced programming or processing features such as AI based or enhanced facial recognition, such as Facial Expression Analysis (FEA) (e.g., mood recognition from a face shot or other photo), object recognition, etc. may be provided by or hosted on one or more remote modules and/or servers (e.g., AI facial analysis, emotional analysis, or mood recognition, hosted on remote server 140, for example). Such programming may include, for example, identification/categorization of photos, objects in photos (e.g., image searches for drinks, tobacco, drug products or indicators thereof), scans of social media postings including image searches and text scanning, etc.

A master program or coordinator (e.g., at Back-End 142) may communicate with the app, user through the app, community, and other programs to gather and further analyze the available data for triggers or trends (e.g., events leading to increased risk of relapse). Such analysis or results may be communicated back to the user in the form of a notification/alert, post on a local feed (e.g., 108), or update of a risk indicator (e.g., 104), for example. Such analysis may be performed by the master program or coordinator at Back-End 142 or performed by specialized routines on one or more remote servers (e.g., 140). The specialized routines may include or utilize API's for various functions related to other platforms or services (e.g., access to social media platforms, retrieval of data from various sources, AI/ML services, etc.).

FIG. 1B is a drawing of an architecture, communications links, and processes according to an embodiment. A user's mobile device 152 runs an app that captures emotional cues from the user (e.g., pop-up query, notification/log-in, responsiveness to queries, etc.). Such emotional cues may be captured, for example, by asking the user to rate his/her current feeling, state-of-mind, or other state. The state may be captured by asking the user to enter a number from 1 to 10, pressing an icon (e.g., emoji) that most closely matches the user's current feeling (e.g., icons 153), selecting a radio button (e.g., button set 154), or other methods. In one embodiment a plurality of sets of selections, for example, four rows of emoji characters each row used to describe different sets of emotions such as, for example 1. General well-being, 2. Anger/frustration, 3. Economics, 4. Control Over Situations/Outcomes, and/or others. Not strictly limited to pure emotions and may include how the user feels about events or circumstances that themselves are not emotions but may, for example, elicit an emotional response or potentially cause actual emotions to crater or peak. The state (or states) may be captured in more than one way and each capture may be used to combine, average or used statistically over time to improve or interpret the ratings. The state(s) may be captured along with other relevant data (settings, recent activities, etc.) and stored in a database, for example (e.g., relational database 154, 200, or other storage, etc.).

Video and/or image responses (e.g., captures) 156 may also be utilized to evaluate the user's current condition, and such video and/or image captures may also be stored and utilized to improve interpretation of later images and/or data captured such as that data captured from the user, the user's mobile device (activities, locations, etc.), or other sources. Such video and/or image captures 156 may be images stored on the user's device, captured at the request of the app, or provided from other sources (e.g., an image from a community member during a recent visit). In one embodiment, the app may request a check-in that includes an image capture, and such image may be compared or contrasted with one or more previous images captured on the same device, posted to social media (e.g., social media 162), or from other sources, for example. The user's willingness to engage the application and complete timely responses (images, questions, or other tasks) is yet another data point.

Previous determinations of relapse states or evaluation of a patient/user were mainly the physician looking at the user, and determining their condition which although would include some non-subjective parameters (vitals, blood pressure, etc.) the overall assessment is mainly how bad the person looks in addition to or as supported by that data, and no two evaluations of the person or the non-subjective patterns were necessarily the same. Here, even if the photo or other analyses are wrong, the results are still collected and maintained with or without subjective analysis and over time become repeatable and consistent, and they provide a basis for future evaluations which will constantly be improved. For example, key facial features may be recognized after just a few episodes that can be the basis for a ‘tell’ that can have significant weight in determining the users relapse progression and/or risk of an immediate and/or ongoing relapse event.

Subjective input or evaluation of a user image may be made by sending the image to a physician or other team member ad asking them, how is this person? The ratings from several team members may be collected and stored for suture evaluations. Such ratings may be maintained alongside other data and be used in an overall determination of the user's level of risk.

Location data 158 may comprise, for example GPS signals processed by mobile device (e.g., mobile phone 152), radio signal data from cell towers, or other sources. A user may grant permission for the app or its associated services (e.g., cloud based services) to access location or other data captured or maintained by cellular service or other providers, for example. Such permission grants may be, for example, specific opt-in acknowledgement captured at app set-up, provided in preferences, rehab or court ordered, etc.

Key Logger 162 may be resident on the user's mobile device and/or a service on cloud 160. The key logger may track a user's activities (e.g., key strokes), and may extend to web sites visited, emails read, application utilized (including input/output from such applications, websites, etc.). The extent to which the key logger 162 (or other programs/processes described herein) extend(s) into various areas of privacy concern, permission to do so may be provided, for example, by specific opt-in acknowledgement captured at app set-up, provided in preferences, rehab or court ordered (e.g., terms of entering rehab or release, etc.).

A social media module 162 may include facilities to monitor and scan social media posts. Posts by the user, the user's friends, contacts, and those the user comes in contact with. Such posts and analysis of the posts may be compared in real time with other data collected to confirm a user's current status or state-of-mind. For example, a user checks-in and provides a self-assessment including a picture, such picture may be compared to the self-assessment score and a comparison of previous photos including recently posted pictures, for example. Such a series of photos may show a changing emotional state that may indicate a level of intervention or other measures to assure the user stays on track in their recovery.

Utilities 163 may include capture and use of telephone, electricity, or other utilities that may provide data reflecting the user's activities or lifestyle. Such data may be captured, stored, and/or processed (e.g., at Back-End 142), and, for example, ultimately used to verify normal or changing patterns of activity or habits of the user. For example, a user that typically maintains a bedtime of 10:30 PM suddenly shows late night or early AM spikes in electricity use may indicate that a welfare check, emotional cur update, or communication from a counselor may be in order.

Payment systems 164 may include credit card, debit card, Pay-pal, Zelle, Venmo or other services in which the user has granted permission for the app (or its associated backend or other services) to access financial information such as purchases and payments. Such data may be compared to other events/data and/or automatically notify the system of a suspect purchase.

Partner systems 165 may include any other systems or collectable data (which may have a similar origin to systems described elsewhere herein) regarding user activities or the user's physical or mental condition (including medical data, club data, golf scores, gym/workout data, biometric data, internet usage/screen time, Netflix or other accounts information, etc.). Machine Learning (ML) module 166 may have access to data collected by the app and/or data from all other systems (each again with appropriate permissions).

Geo-Spatial Analysis module 168 may provide analysis of the user's location. For example, identifying the user's specific location and a probably activity at that location (is the user in a store, is the user in a liquor isle of the store?—a red flag alert warranting an immediate contact or intervention).

The machine learning module 166 may be set-up to search for patterns in the data including patterns applicable across the user spectrum, specific classes of users (e.g., alcohol issues, tobacco issues, drug issues, gambling, gaming, for example), and for the specific user as an individual. Such patterns may include, for example, identification of events or patterns of conduct across one or more partner or other systems that may identify a changing mental state, changing living conditions, changing habits, etc. Such changes may include, for example, identification or indications of a potentially more depressed state and/or a more elated state, each of which may reveal a situation or circumstances where an intervention or other contact with the user is warranted.

The machine learning module may vet rules by running them against large data-sets collected over time (e.g., data-sets related to the user, and/or larger datasets such as a class of users, and/or all users). Such rules may be weighted based on, for example, an accuracy probability for each class of users and for this particular user. Such rules and/or their weightings may then be utilized by Artificial Intelligence (AI) module 167 to make decisions regarding a user's state or probability of a relapse and/or need for intervention or other contact.

Analysis & Notifications module 169 may provide programmed notifications based on pre-determined events and/or notifications based on analysis performed by one or more modules (e.g., Artificial intelligence/Machine Learning modules 166 and 167), and such analysis may result in, for example, identification of an issue (e.g., serious change in the user's emotional state, root cause identification, etc.). Notifications may include, for example, pop-up requests for emotional cues, request for specific information from the user (are you on track today?), notifications corresponding to analysis, and/or a notification to a counselor or other support member which may include contacting support members (e.g., network/community) based on level of experience and/or proximity to the user.

Such notification may include all the particulars of the user to be contacted, including name, photo, location, and fast access buttons or highlighted fields (e.g., phone number) to make immediate contact, and such notifications may include suggest approaches or contact methods (e.g., “user more likely to pick-up if you text first”). The counselor or other support member (e.g., personal sober community outreach 170) may be tasked with making a personal contact (e.g., phone call) and then possibly included in a physical/actual intervention group depending on whether the user can be reached and/or responses received).

Such activities and any escalation/follow-up may proceed according to a protocol that may be organized, arranged, and implemented by the system (e.g., Back-End 142 or Analysis & Notifications 169, for example). Other modules, such as programs resident of the user's mobile device may be programmed to participate or coordinate such activities as well. Such organization may include, for example, sending alerts to 3 or 4 of the nearest located team members. The platform (or backend) may in real time track locations of team members or send requests for locations prior to sending alerts to the closest team members (alternatively any team members available may be notified). The alerts may, for example, be first filtered by schedules (e.g., access to team members schedules) to assure they are not in a blocked out/busy time frame, and send alerts as to any of the current need, location of the user, circumstances of the alert, etc., and await for an affirmative response that the team member is available (and preferably immediately on his/her way). In this manner the platform puts together a response team and tracks who has responded and is in action. In one embodiment, the platform continues to track the locations of team members and may, depending on the circumstances, notify the user with encouraging messages such as “Team member 7 (name) is on his way . . . only 7 minutes away,” and may include a display of team member 7's progress toward the user. The platform includes intelligence and decision making power in cases where alerting the user as to arrival of the team member might not have a positive effect and suppress those types of messages. In one embodiment, a drone is dispatched from a central location to interdict the user, providing a distraction and delivering encouraging messages such as a video call on a screen attached to the drone after landing. In one embodiment, the drone may locate the user's vehicle and physical put itself between the user and the vehicle to impede progress of the user toward the vehicle. In one embodiment, a physical intervention is accomplished entirely by drone (e.g., physically flying the drone (e.g., via entirely computer implemented flight instructions) to the user's location and communicating with the user via the drone).

One or more analysis may conclude a risk level that is elevated but not imminent, and in such cases, automated messages or interactions with the user such as to notify the user about the elevated condition and/or look further into potential issues without immediate or direct support member contact may be utilized (e.g., low level issues). And the same or similar app/user interactions and messages may be utilized in conjunction with team member notifications, communications, and/or mobilizations. Team member notifications (including team leader assignments), group organization messages, etc., may also follow a protocol that, for example, requires an affirmative response accepting responsibility for taking necessary actions in response to the notification—absent which the notification may be re-routed to other team members (or other groups/teams/contractors if none are available) until the appropriate team or team member is identified and ready to respond.

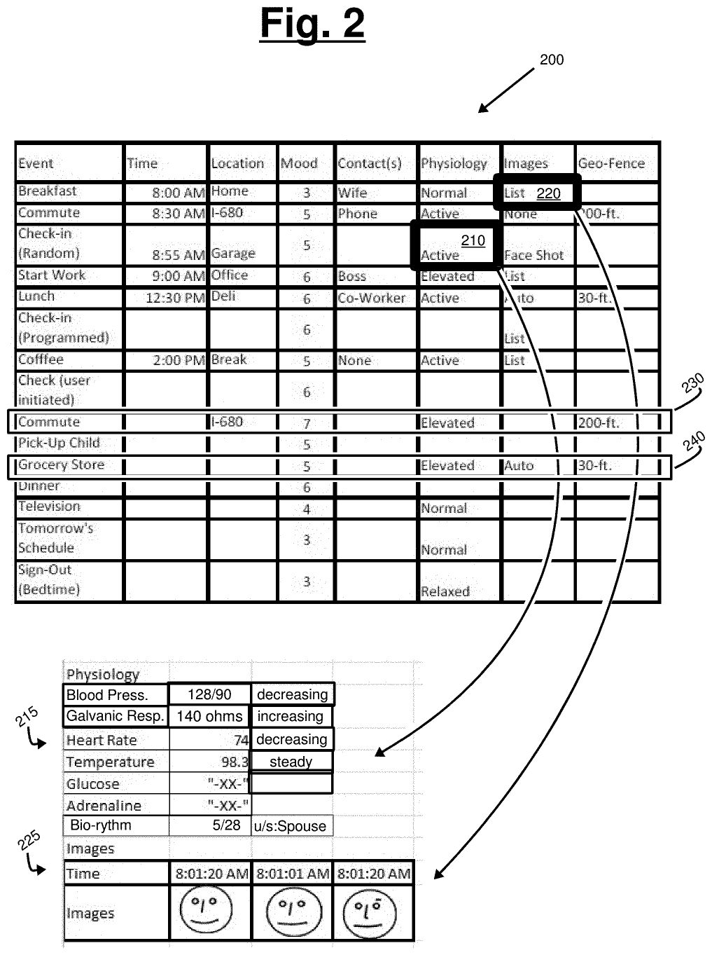

FIG. 2 is an example relational database 200 organized to keep track of recovery and other relevant data according to an embodiment. The database may include various data points, such as, for example, routes, frequently visited stores, etc., list of known danger spots (bars, dealer locations, etc.), purchase histories, biometrics, and other data which may be correlated or used to correlate against other factors including use of the same to determine a probability or potential likelihood of a relapse or other event. One such correlation may be, for example, recognition of an event or conditions that could trigger a relapse which is then prioritized for a response.

Such response may be an interaction with the application, a direct call from a healthcare professional or friend, and/or an immediate response team intervention which may be in the form of taking the individual out of the situation by force if necessary (non-revocable and approved in advance, for example). Other responses may include stand-off observation but ready to intervene, for example. Such responses utilizing team leaders, team members, or others (e.g., contractors) may be organized by the application (or backend) by, for example, first sending notification to the team leader (or selecting a team leader, e.g., via the above discussed protocol), and then filling the team as needed (e.g., as suggested by the application or backend, or as specified by the team leader) using a similar protocol.

In the example database 200, a subset of potential data is shown, including time/location stamps, Mood indicator (which may be a predicted mood or answer from an emotional cue), a contact if applicable (e.g., who is the user interacting with at this time/location), Physiology, Images (which may be application requested images or images taken automatically, and geo-fence data (e.g., is the user near, approaching, or on the way to a geo-fenced location (e.g., liquor store) or location having a geo-fence (e.g., Safeway—O.K. to enter Safeway, but beer and wine aisles are geo-fenced as impermissible to enter).

The database may include parameters of each user's geo-fencing bounds as some users may be held to more stringent geo-fencing policies (e.g., in some cases entering Safeway may not be permissible even though aisle level geo-fencing is available). Portions of the database, such as geo-fencing parameters may be copied and stored on a user's device and interact with GPS signals or locations to produce pop-ups, notifications, physical alerts (e/g/, phone vibrations), sounds (e.g., alarms—including, for example, loud tones that are not user configurable and may only be turned off by exiting the geo-fenced area). The geo-fence violation itself is logged into the database and may, for example, be sent via alert to the user's counselor or a team leader and may be the basis for formation of a response team. Geo-fence violations, like other events noted and/or stored in the database, may then be compared or correlated to other data collected in or around the same time, such as one or more physiology factor, emotional state, etc. Such comparisons or correlations may be done with the aid of ML/AI module 166/167 and provide a basis for future predictions, alerts, or warnings.

Various entries relate to other tables or databases, such as, for example, physiology entry “Active” 210 (a summary of physiological data) points to physiology table or database 215 which may provide, for example, a more complete picture of the user's physiology at the date/time stamp. The physiology table may include blood pressure, heart rate, temperature—and other items that may be determined from sports monitors or other equipment (e.g., medical equipment at a doctor's office), watches (e.g., Apple watch), gym equipment, and other devices). The physiology table may include an entry for biorhythm or stage of a hormone cycle for the user or that of the user's partner. The physiology (as a whole or individuals data points) may then be correlated to other events at/around a same time, and such correlations may be used in future predictions or indicators of emotional health, for example.

In one embodiment, the physiology table includes entries from EKG, ECG registered devices and/or galvanic skin measurements (which may be part of external devices such as watches, rings, or electronic patches, or may be an implanted device, for example).

Images entry 220 may be an image, data about an image and/or a list, for example, of multiple images and/or data. Such images may have been taken in response to a query from the app (e.g., emotional cue query that included a request for one or more snapshots). The various images may be time stamped and stored for analysis. Data about the images may be extracted from AI routines or services that note significant messages, tones, or emotional content of the images and stored (and then used in any contemporaneous or past event evaluations, for example).

The database as a whole stores this and other data to be processed and reviewed for patterns and other information relating to the user's emotional state and potential for a relapse. Looking at the database as a whole, and objectively, it can be determined, not knowing any other information about this user, that a more likely time for an event appears to be at the Commute entry 230—which shows a combination of elevated mood, no contacts, elevated physiology, and relatively close proximity to a geo-fenced location (200 ft.). Without knowing more, it may be prudent for the system to send a notification or alert to the user's support network for an outreach. Grocery store entry 240 (not long after the commute entry) includes some of the same potential warning signals/combination.

That being said, such analysis is likely more meaningful when considered in context with a history of such entries collected by the app over time. The history may show such events have occurred under similar conditions without impact and may (at least at this time) be set-aside (but may still warrant a follow up email telling the user s/he successfully navigate a rough commute or potentially dangerous store event. Alternatively, the history may show past relapse events under similar conditions warranting an immediate and direct intervention.

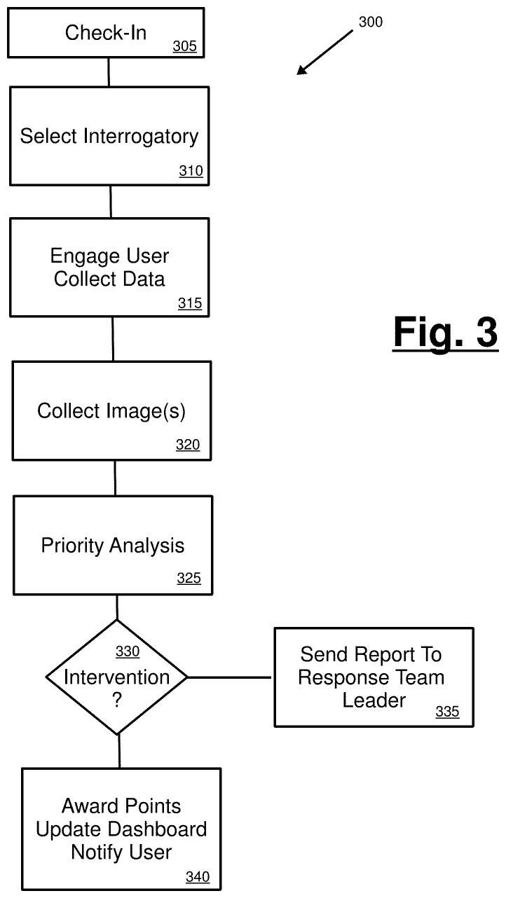

FIG. 3 is a flow chart 300 of an exemplary data collection process according to an embodiment. The data collection process may take the form of collecting biometrics, travels, events, and/or emotional cues. For example, at step 305, a check-in is initiated. A interrogatory may be selected which is, for example, any of a request that the user rate his/her emotional level or mood on a scale of 1-7, select a emoji reflective of their mood, pick a color that describes how they are feeling, or others. The interrogatory may be randomly selected, may be consistently selected for a period of time (e.g., same query for a week, then switch to another query), or may be set to a query that user most favorably relates to. The selected interrogatory is then used to engage the user and determine their emotional level or mood or current mental condition (step 315). The interrogatory may include the collection of images (step 320) that may be taken by the user. In one embodiment, images are acquired by commandeering the user's mobile device camera and actively looking for a face shot of the user and capturing it when it is available. Such a routine may be utilized to collect face shots at other times which may relieve the user from that duty at least at times when it would be inconvenient to capture an image manually. All such images may be processed with FEA or other techniques to determine the user's current state (emotional state), and then, for example, stored in the database along with the determined state or other metadata. The determined state may also be supplemented, modified, and/or cross checked against other contemporaneous data such as location, recent activities, biometrics (e.g., vital statistics, GSR, etc.), compared to previous emotional states of the user (and previous contemporaneous data), and compared to similar data of other users, for example, and may be combined for a combination of factors relative to location or other circumstances, for example.

The information and images collected may be analyzed to determine the user's current emotional level or mood or mental state. Such analysis may include comparison to biometric data collected as part of the check-in and/or review of biometric data compiled over time, and/or review of biometric data compiled over time under similar circumstances (e.g., reviewing previous times the user has been at/near the user's current location and comparing to the user's biometrics at those times to current readings). Such analysis may be, for example, a priority (step 325) or real time analysis to support a decision (e.g., decision to intervene—step 330). If an intervention is warranted, a report or notification may be sent to the user's sobriety network outreach community (e.g., Response Team Leader (step 335). The notification may be managed by the system within the user's network to avoid conflicts (e.g., reach out to individuals in the user's network that are appropriately trained and available at that time, for example). If a selected or nearest trained member (e.g., team member and/or team leader) is not available (e.g., no response, or declines), a next most qualified or next nearest member (or members) may be contacted.

In the various instances where a team needs to be assembled the team leader and team members are contacted by the app/backend noting the particulars of the situation, user's history, etc. If the team leader (or other member) is available, a positive response from the team leader (or other member) acknowledges receipt of the notice and acceptance of the responsibilities of being part of the team. The team leader is accepting management of the team and committing to taking action. The app/backend handles many of the traditional team management functions (e.g., sending team messages, collecting/organizing responses, preparing team lists, managing calls/video conferences, etc.) so the team leader can spend more time preparing and planning for the necessary steps contacting or intervening for the user's benefit and insuring the response follows established procedures for the intervention or other task.

In one embodiment, the team leader is asked to provide additional information as to the plan of action for any response. This may be, for example, noting that a group call is being organized, or that an intervention is planned, and/or both, for example. Some standardized actions may be selected on a menu such as call, organize intervention etc., and such selections may, for example, fire an API trigger to another system or module (or a disparate system) to handle that task or portions thereof (e.g., find and organize volunteers for an intervention). Such API may be, for example, a process outside of Prelapse (the app) and may be hosted by another entity altogether.

In this manner the system initiates and helps manage the response team to most effectively and quickly provide an appropriate response. In cases where no qualified members are available, a volunteer may be contacted to intervene along with phone support from a qualified individual. Such interactions may be recorded on the user's device (and the volunteer's device(s)) for later review, discussion, and process improvement. In one embodiment, if members of the user's network are not available, trained volunteers (or paid response teams) outside the user's network may be contacted. Thus, the system may automate and manage contacting volunteers/personnel in a tree-like hierarchy to assure that the user gets the most qualified contact/intervention available as needed to prevent a relapse or lessen the impact of a relapse in process as soon as possible. And of course, not all interventions will require larger manpower and lesser event may be managed, for example, with a phone call from a therapist, case worker, or other individual.

The system may further facilitate collection of information regarding the user's emotional state by requesting information about the user's emotional state from those with who the user interacts, including, for example, those in the user's sober network, friends, co-workers, health professionals, team members (e.g., requesting an evaluation after an intervention), and others. The system facilitates collection of the emotional information and also facilitates interactions with the user, such as interventions or in-person check-ins (scheduled and/or random) if applicable (e.g., part of a user's rehab release plan, order from a doctor, therapist, rehab center, or court, etc.)—all of which may include a follow-up from the system/app to the team member or other professional, etc., requesting an evaluation or impression of the user's emotional state at the time of contact. Such evaluation may then be compared to information that the app has collected in/around the same time frame and predetermined time periods before and/or after the contact to provide additional insight as the user's emotion state and probability of relapse or other event.

At certain times, it may be appropriate to award points or suggest the user make a diary entry (the system may include facilities to quickly add diary entries (e.g., automatically make a suggested diary entry if accepted by the user) (step 340). Such points may, for example be added to a total points maintained on a homepage or dashboard of the app and provide a form of reinforcement.

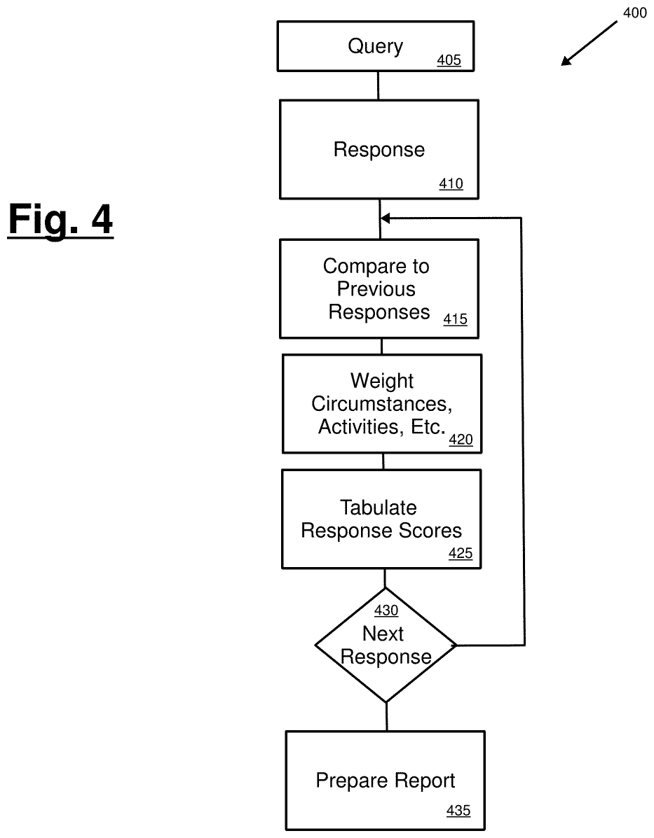

FIG. 4 is a flow chart 400 of an exemplary analysis process according to an embodiment. The process may be set up for analysis that may include, for example, identifying events or potential events, and issuing notifications/alerts to the various stakeholders as, for example, enumerated in a preferences function of the application or built-in by design for a program. The process may begin by providing a question, quiz, puzzle, or other query (step 405) and capturing a response (step 410). The response is compared to previous responses to the same, similar, or similar types of question or query (step 415). If the answer is significantly different from previous similar queries, a red flag may be raised for further analysis/query. The response may also be weighted based on, for example, known circumstances, such as recent activities, biometrics etc. (step 420), and a cumulative score or total may be maintained (step 425). The queries or other assertions/responses may continue (step 430) and associated comparison/weighting/tabulation until complete and a report or score may be provided to a decision module (e.g., step 330, FIG. 3).

Accordingly, the various embodiments include multiple layers of data gathering capability, some that require a user's permission—others that are publicly available (such publicly available data may include social media, local and national news cycles, etc.), and such data may be organized and analyzed (e.g., as described herein or in other ways) to establish relationships and patterns with respect to the various data points. Further, such data may also be collected from other individuals in the user's various circles, and particularly those whom the user interacts with on a regular basis (but may certainly include others that interact tangentially or more remotely). For example, data may be collected on additional individuals is from those in the user's sober network and at all levels of influence.

The user's sober network may include those identified by the user while using the app, and once linked to the user may be sent a download link for the app (or an adjunct data collections app) along with permissions including opt-in for any collections that may be desirable. For example, social media posts, location tracking, and face shot analysis—a similar or subset of data collected with respect to a user may be collected on/about individuals in the user's community, sober network, or other groups that, for example, interact with the user. This data is then analyzed/searched for patterns with respect to the user (including FEA of the user and the other individuals), the user's activity patterns, and emotional state, which may ultimately reveal patterns or circumstances that are related to or that potentially affect the user's emotional state, identify triggers, etc.

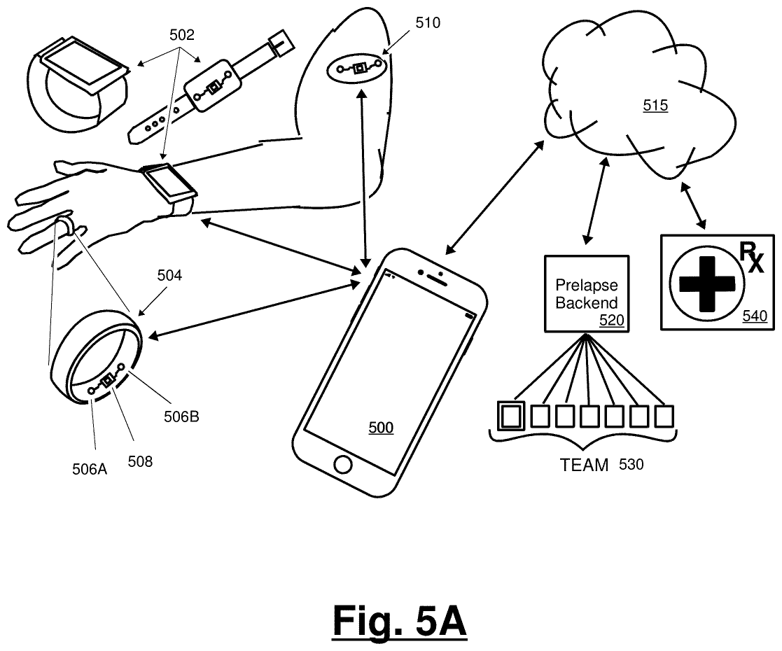

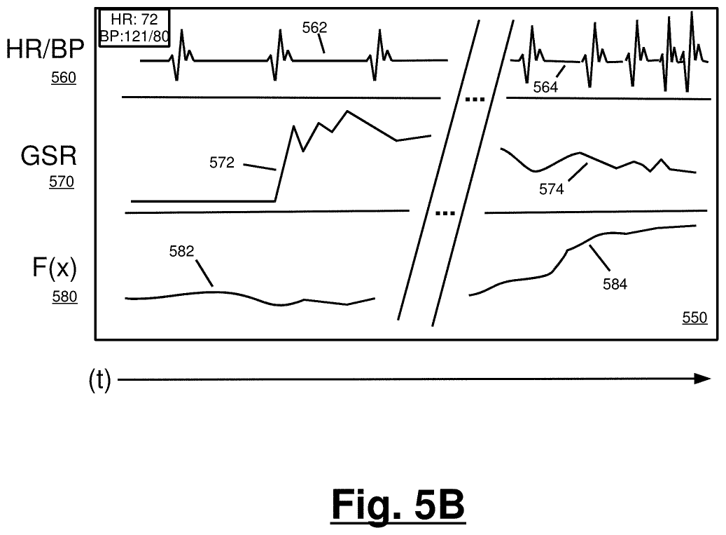

FIG. 5A is a drawing of biometric collection apparatus according to embodiments of the invention. A wearable device (watch form factor) 502, a ring 504, and/or a patch 510 may be worn by a user. The devices include apparatus for measuring biometrics such as heart rate, blood pressure, Galvanic Skin Response (GSR), and others. The device may include, for example, a plurality of electrodes (e.g., 506A and 506B), and a processor/communication module 508. Other hardware including LEDs, photo-sensors, RF, and/or audio capabilities may be included as desired to implement one or more biometric measurements and communication with the app (e.g., installed on mobile device 500). The electrodes, may, for example, be configured to measure resistance, electric current, or other properties and the processor may be configured to calculate GSR along with any other biometrics the device is configured to measure, GSR (e.g., electro-dermal activity is the property of the human body that causes continuous variation in the electrical characteristics of the skin). The Galvanic Skin Response (GSR), which falls under the umbrella term of electro-dermal activity, or EDA, includes changes in sweat gland activity that are reflective of the intensity of a user's emotional state, or emotional arousal.

The wearable devices may be placed in other locations, and implantable devices may replace or supplement any measurements (e.g., an implanted ECG, which may have been implanted specifically for use with the app or for other medical reasons, the data from which may be accessed and forwarded to the app on mobile device 500). If an implanted device has other medical functions, the app my facilitate transfer of the data collected to a medical or RX facility 540 via cloud 515, and/or a physician with knowledge of the user's Prelapse monitoring may request access to all data which may also be forwarded to facility 540. Further if the user already has a monitoring devices, external or internal (e.g., implant) for other purposes, with appropriate permissions the data from existing devices may be collected and forwarded to Prelapse.

Facility 540 may be a rehabilitation (rehab) center or program which receives regular updates and maintains current status on a plurality of users and has access to any of the users' data on the backend 520, mobile device 500, and/or other devices. The data collected is ultimately placed, for example, in a database (e.g., relational database 154, 200, or other storage, etc.) and used to calculate a current emotional state of the user, probabilities of relapse events, and otherwise facilitate decision making related to features of the app and its associated processes (e.g., preparation and organization of team 530, when needed).

FIG. 5B is an illustration of collected biometrics data and an example of how the data may correspond and be utilized according to an embodiment. The illustrated biometrics comprise a subset of data that may be collected and are provided as an example of the type of data collection, correlation, and processing that may be implemented. Heart Rate & Blood pressure data 560, Galvanic Skin Response 570, and F(x) which may represent any of data or combined data collections, and here specifically providing an example of a combination of all other data collected and processed to determine the current emotional state of the user and risk of relapse but excluding GSR and HR/BP for purposes of this example.

As can be seen in segment 562, a user has a steady and normal heart rate. In a same time frame, the Galvanic Skin Response 570 has a significant change (increase) 572. Such an increase may be, for example, an increase in resistance measured at the surface of the skin (other measurements may be utilized), for example as measured between electrodes on an appropriately configured watch, ring, patch, or an implanted device. The increase may be, for example, from 150 ohms to 250 ohms, or a percentage jump of, for example, 50% or more, or a steep jump in GSR from any level—any or all of which indicates that the user is responding to some stimuli which may be meeting someone, being startled by an event, and/or passing by an outdoor restaurant and smelling alcoholic drinks (even if not consciously aware).

Like other data collected and monitored, significant changes are important for the app to know the circumstances causing the change. All other collected data at about the time of such changes (e.g., location, other biometrics, activities/exercise, and sleep/work/eating cycles) may be flagged as a set of data representative of a significant change. The change may also trigger an automated response from the app (or call from a team leader) that may, for example, ask the user what his/her current situation is.

It is important to note that in the same time frame, the user has a normal heart rate 562 (heart rate has not yet reacted significantly to whatever the stimulus was), and the F(x) segment 582—processed data from all other data collected shows only mild variations indicating all other factors appear reasonable.

Moving ahead in time, the GSR begins to recover 574, but all of the other factors F(x) 584 have increased significantly along with the user's heart rate 564. In fact, the user's heart rate and other factors clearly indicate the user is experiencing some sort of turmoil and may likely be in an emotional state conducive to relapse. Such conditions trigger an immediate app response and formation of an intervention team.

An important aspect of the above example is that the GSR showed potential of the turmoil before the other factors and other biometrics kicked in to indicate that turmoil. The GSR may have responded before the user was even conscious of the potential turmoil. For example, alcohol odor wafting from a restaurant that may be sensed but faint enough that the user does not consciously recognize it, the GSR will react (at the sub-conscious level). Accordingly, along with conscious reactions, embodiments of the present invention take advantage of the sub-conscious reaction shown in galvanic skin response which is a data point from which to evaluate a user's emotional state and to correlate with other data for current and future emotional state predictions.

In this example correlation may include, for example location—the location of the user at the time of the GSR response may be noted as a potential trigger point and evaluations of the user's state may be more critically evaluated when passing this location in the future. Warnings/Notifications to the user and/or others (e.g. team leader) may be issued when the user is on a route that may include the location or similar locations. Maps and routing as provided elsewhere herein may take the location into account. A geofence may be established around the location. Other sensing devices may be placed on high alert or threshold levels reduced when near the location (more likely to issue team/team leader notification).

Correlations to other data may also be made and appropriate notifications when similar conditions occur. Accordingly, such correlations may be made with any of the individual data points or any group of data points including any of the data points discussed here and/or other data points that provide any information whatsoever in determining a user's state of mind, emotional state, probability of relapse, etc. (including data points with respect to such states of family, friends, and associates of the user).

Further, the GSR data collected is maintained and along with the other data points provided to Machine Learning (ML) and Artificial Intelligence (AI) routines to correlate the data in every possible way and look for patterns across the dataset. The GSR being a response that can provide an early indicator of downstream stress or emotional conditions conducive to relapse potential and a key to identify such conditions before a full-blown relapse occurs.

It should be understood that the illustration of FIG. 5B is just one example of a possible GSR and other data of an individual user during essentially one time period. Data collections at the same time but different location (or vice versa) under similar circumstances has a probability of a similar result but could be very different. Accordingly, the collection of data over longer time period will likely increase the possibility of correlations of current data providing better information. This includes correlations over the larger dataset of all users which may be applied to any specific user, as well as the user's specific data.

Further, the collection of data including GSR for a large number of users increases the data pool to the extent average responses to known conditions usually has at least some correlation to current events for any particular user. Further yet, the differences between the general population response (e.g., emotional condition) and an individual itself can be statistically meaningful when compared to an individual user's emotional status a relatively few number of times and can quickly identify triggers when the user's status changes significantly.

GSR measurement may also be specifically noted when the user is performing actions of known quantities. For example, measuring GSR while the user is taking an emotional que questionnaire (e.g. selecting feelings, answering questions, etc.). Veracity of the answer might also be implied (e.g., if all other conditions point to low emotion state (high relapse potential), and the user responds that s/he is happy and things are going well, but the GSR indicates agitation (especially if such responses were confirmed earlier) then it may be appropriate to provide a further inquiry and/or initiate a team response.

The GSR may also be measured at precise points of any particular activity, such as, for example, while taking a selfie, texting certain individuals (permissions may be set such that Prelapse reviews who the user is communicating with (e.g., text, voice, email, conference call, zoom, etc.), and the GSR may be evaluated for how well the user is reacting internally while interacting with these individuals. Such reactions may be mapped to each line of text as drafted, sent, and/or received/read.

In one embodiment, the GSR readings (or any or all other data points) are collected over time and correlated to other data to provide a baseline of the user's GSR dependent upon location and other circumstances (e.g., the correlations to other data). Those collected values and circumstances may form a baseline and a standard deviation having upper and lower limits which effectively show the user's range of “normal” state of being under those circumstances. In the case where a user's GSR readings either fall short or exceed that normal range the platform may trigger various alerts. Since each user will have their own responses, the GSR alert points will vary one user to the next, and any individual user's GSR alert points will vary according to the user's location (or other circumstances).

In this manner, each individual user's circumstances, and normal responses are accounted for. For example, in a situation where a user registers a high or low GSR, but the user is at the gym or track, the user's data will show in that location a high or low GSR is normally encountered and no alerts will be triggered. On the other hand, if the user is at home, near bedtime, and typically winding down with a low GSR, in that case a high GSR reading would be looked at more carefully and likely the platform would at least inquire as to the user's circumstances, request a self-test, or trigger other alerts.

Further, as the previous example shows, in addition to correlating the reading to location, they are also correlated to time of day or other factors (e.g., for this particular user, being at home is not necessarily indicative of a low GSR, but normally is at bed time). In one embodiment, all other collected data is cross-referenced and analyzed with respect to location and time along with all other data points collected. Each combination of data-point (e.g., GSR) vs. location, or data point (e.g., GSR) vs. data point (e.g., vitals) may be analyzed and provide a standard deviation or score to add or subtract from a relapse score being calculated, for example.

In one embodiment a tension or stress score is determined by taking images of the user, comparing the images to baseline images, and determine a score. The score may comprise, for example, a deviation from baseline of one or more lines marks or crevices on the user's face and may be quantified as a percentage increase in depth and/or width of the lines.

In one embodiment, a stress/tension score is based, at least in part, based on lines on the user's face. Such lines include the frown line, crow's lines, and others. The depth or width of such lines may be determined via echo location (sound ranging and mapping) image analysis, or other techniques. For example, taking images of the user from different angles (e.g., images from a dual lens/imaging camera on an advanced mobile phone), identifying the line (e.g., object recognition), and geometrically calculating a depth of a frown line on the user's forehead (e.g., determining distances to a lines ridge and a distance to its trough bottom). Such measurement and calculation may be made a prescribed predetermined spots along the frown line, or may be multiple readings at random locations averaged.

Such calculation may be done at multiple times and averaged to determine a resting state (or low relapse score state) baseline of the user. For example, an average 1 mm depth frown line with a standard deviation of 0.1 mm determined from multiple measurements.

In one embodiment, the frown line depth is calculated at various times (with or without the user having direct knowledge that it is occurring) and compared against the baseline and its standard (std) deviation. If the frown line increases 50% in depth over the baseline (or above baseline+std deviation) it can be correlated to an elevated relapse risk score, if it doubles, the user may be at risk, and if it triples or more a relapse event may be a certainty.

The levels at which user may be determined to be at risk (above noted at 50%, 2×, and 3×) may be determined via a statistical average of a pool of users compared to their relapse scores. In another embodiment, the risk levels are determined by comparing multiple evaluations of the user over the course of days, weeks, or months (and in fact may be an on-going process continually improving the analysis). For example measuring the user's frown line depths at different times and determining a relapse score at the same or corresponding time frames. In yet another embodiment, the relative risk levels are determined by weighting the users frown line over time corresponding to relapse risk together with weighting the pool of users (and such weighting may vary by types of users in the pool) as to how the pool corresponds to this particular user. In such embodiments, the users score relative to his/her frown line is a combination of the general population of users and specific knowledge of how the users' frown line is reflective of the user's current relapse score.

It also can be noted at various depths the certainty of the predictions. For example, an individual user that has been monitored over time at various frown line depths will typically show a wide range of variances, but may show some particular points where the frown line is highly accurate. For example, the user may show variances within some deviation that the frown line is more or less indicative (or evidence) of the user's relapse state and may therefore be afforded a 60% weighting in most categories. However, the same user may consistently register a 2× frown line depth as being in an irritated or insecure state and may with accuracy, on its own, be indicative of an extreme relapse event risk and be weighted at, for example, 90%. Such weightings may also be increased or decreased based on other indications for example, other facial lines, heart rate, blood pressure, etc., each of which may analyzed and categorized in a similar manner as discussed above.

Further, the accuracy of such measurements is increased when all available indications are similarly recorded and vetted for the pool and the user individually. Such measurements may be searched for consistent combinations of indications that are highly accurate in determining relapse score and risk of this particular user. For example, the combination blood pressure over 140, 2× frown line, and dilated pupil may be a combination that is nearly 100% accurate in one individual, 80% accurate in a group of like individuals, 50% accurate for the population (or pool) as a whole, and perhaps only 25% accurate for pool members of different dispositions (e.g., different age bracket, different sex, etc.). The various accuracies are determined by collecting the indications and relative scores over time under different conditions.

The weightings may be determined, for example, out of a score of 100, such that each indication gets part. For example, with 4 indications (e.g., heart rate, blood pressure, frown line depth, and an activity score), each may be afforded points which is weighted by how well correlated the indication is for the individual. For example, heart rate score may be determined by a rule that awards 1 point for each beat per minute above baseline, which is then weighted based on one or more other circumstances. That score may then be added to the scores from the other indications (e.g., also according to rules) to determine an intermediate score. The rules for combining the score may include one or more short-cut or sure-thing indications or combinations of indications that have been shown over time to be highly accurate (as discussed above).

In one embodiment, all operations are within a genus of recovery systems having the common characteristics of comparing current trending psychological physical indications with past activities to determine a probability and the prediction of relapse event, finding a patient in relapse but before the event. The common characteristics define parameters of which the user is evaluated for relapse probability. The common characteristics define specific combinations of points of evaluation and analysis based on recorded history. Alternatively, as opposed to prediction of a specific relapse event, a point is determined on a progression of relapse which the individual user is experiencing.

The intermediate score may be further adjusted based on, for example, a historical weight of past relapse scores and transitional parameters associated with the user and/or similarly situated individuals under similar conditions wherein relapse scores and transitional parameters directly attributable to the user are weighted higher than scores and parameters of similarly situated individuals which themselves are ranked and weighted higher or lower depending on degree of similarity to the user. Such further adjustments may be, for example, a second set of rules.

In other embodiments, facial features such as under-eye darkening, mouth or lip posture, eyelid position, head pitch angle, or other features may be similarly analyzed for shape, color, or simply just any differences from a baseline for those features and correlated to a relapse score at the time the features are recorded.

The embodiments effect an improvement in recovery systems because they provide immediate information and analysis that can be acted on before a full blown relapse event (or a relapse has progressed too far without action being taken. (e.g., into drinking or substance use). For example, determining a level of relapse, a rate of relapse progression, a non-leveling of relapse progression or potential. The embodiments effect an improvement in the field of recovery systems and provide new or different tools from which an analysis, prognosis, or treatment options may be considered.

In various embodiments, meaningful requirements are established for evaluating user indications including combining user indications as a function of a sequence of indications corresponding to one or more factors in their appearance, intensity, and longevity. In another embodiment, those same indications are further considered as a function of circumstances in which they are detected and/or in combination with other indications. In yet another embodiment, the same indications (and/or the further considerations) are yet further evaluated as a function of those indications (or combinations) compared to values of the same indication in similar past situations.

For example, in one embodiment, the application detects a user's average proximity to one or more establishments (e.g., bars or other locations known to serve alcohol and/or sell alcohol). The average proximity may be established as a function of time or day and/or a function of typical daily activities such as going to work, grocery shopping, etc. And although meaningful information or analysis may be established from that alone (such as upon determining that one day a user came within 600 ft of an establishment s/he had previously frequented may set off an alert), various embodiments go further and compare that proximity to other information such as any of the physiological and/or psychological conditions being experienced by the user at the same time or within a predetermined amount of time prior or after the user made the close pass to the establishment.

In one example, the proximity is compared to the average heart rate that occurs at the same time of day, same type of day (weekend, holiday, workday, etc.). So, for example, the user entering within a predetermined distance of a previously frequented establishment, and a sharp increase in heartrate, and other sensors not indicating any reason for the increase (the user is not running, the user is not at the gym, etc.). In one embodiment, the increase in heart rate may be evaluated over time such that an on-going raise of heart rate may be evaluated in the same manner as a sharp increase (e.g., heart rate compared to proximity over period of time, such as a continuous or stepwise increase in physiological conditions as the user gets closer to the establishment). Accordingly, the embodiment may be more broadly defined as a sequence of one indication corresponding to changes in another indication. Upon establishing a change or elevation (e.g., heart rate in this example), the process may then look to the length of elevation, and, for example, if transitory, may be disregarded or used as a minor influence (however, depending on other circumstances and the individual's history, such indication may be promoted to a more significant event). If of moderate duration, may also be disregarded if not combined with other indications. If other indications are also present (e.g. GSR response, breathing, eye movements, etc.), again compared against a baseline that may normally vary according to time of day or location, moderate duration may be elevated in importance.

Such analysis may be traded with one or more other indications, for example, GSR may correlated to establishment proximity, etc. In one embodiment, if an increase in GSR is moderate, other indications may be more critically considered, such as for example, increases in eye movements (e.g., as detected via image analysis) that normally might not trigger an alert because they may be within predetermined allowable variance, but above average for the location time of day, etc., may then, in combination with the GSR measurement trigger an alarm. In one embodiment, two or more further indications above average trigger the alarm, and further indication may then raise the level of alarm (e.g., raining the alarm from an automated response to a phone call, and then yet further to full intervention).

In another example one or more of the indications (heart rate, GSR, etc.) may be compared to a psychological evaluation such as the user's current self-test. In another embodiment a trend in self-tests is evaluated and provided, for example, a score weighted toward the most recent test but also including a trend such as increasing or decreasing over the past hour, day, week, etc. that may add or take away form the final score.

In yet another embodiment, weightings of the various indications may be performed based on past performance. Some users may be determined to have one or more indications having significantly more predictive value than others, and those indications may be weighted higher in any analysis. For example, a user who has had several past relapse events for which data was collected may show that amongst those events heart rate and declining psychological scores have consistently preceded the events and those factors would be weighted higher than factors that might have been present in one or more but not all recorded events, and factors which were not present in any of the events may be weighted lowest. However, absence of such factors can themselves be indications.

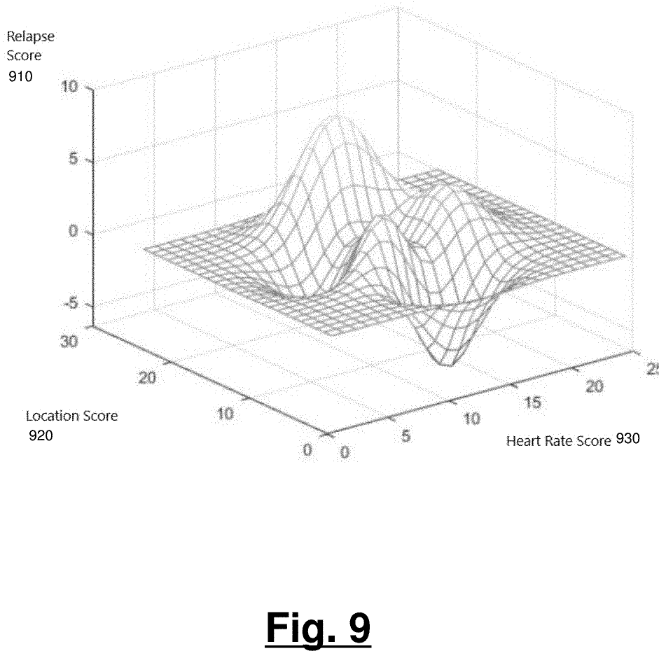

In various embodiments, the analysis of indications alone, in combination with other factors, and then further in combination with comparison of past similar situations in which those indications have been record is a multi-dimensional analysis, such as illustrated in FIG. 9. Here, for example, heart rate 910 and location 920 may together provide a relapse score (e.g., 930) that may trigger one or more alerts, and may be visualized on a 3D graph of location, heart rate, and risk. The location score may be, for example, proximity to an establishment such that 1 point is awarded for each 100 feet less than 1,000 yards a user approaches an establishment. The points may preferably be calculated geometrically such that the last 100 feet are calculated at a rate that is a multiple of the previous 100 feet and so on, such that, for example, the last 300 feet may account for more than 50% of the score (in some embodiments, the last 300 feet may account for 70% of the score, in other embodiments, the last 100 feet may account for 50% of the score). In application then, for example, a location score of approximately 25 (e.g., indicating a user is close to an establishment) and a heart rate score of approximately 15 (elevated with respect to current physical activity or other conditions) produces a high Relapse Score triggering a high level alert or response. However, the same Heart Rate or Locations can produce significantly different scores and responses from steady state (e.g., normal or slightly elevated check-ins, such as Location 25/HR10) to low relapse potential (e.g., HR score 15 and location 5).

Additional dimensions of the graph may be added for history at the particular location, heart rate, or any other factors, meaning that, for example, for this particular user, over time, this particular establishment did or did not participate as a significant factor in the user's addition and may be accorded more or less weight in that regard. Such analysis may be further dimensionally characterized, such as by the time of day, day of the week, or other circumstances in which the user passes by the location (or type of location). As such, the above graph may be shown to morph over time (either according to the particular user or the generalized pool of data from other users) by time of day, day of week, season, holiday, etc.

If for example, this particular location has not been an issue for the user, but has been for a large percentage of other similarly situated individuals, the relapse score may be increased. Such increase may be moderated (e.g., have a lower or even negative weight) in the case where the user is consistently involved in a predetermined set of one or more locations to the exclusion of others. Such location may be the user's home, a bar, restaurant, social event, annual parade, fair, etc. And such locations would also carry significantly higher weights and if breaching a predetermined threshold score (e.g., 8 or 9) may trigger the highest level alert. Such threshold may be, for example, going outside a standard deviation of the user's normative activities, or normative activities for this location or other parameters such as day-of-week, time-of-day, etc.

The score, such as any point on the graph as calculated to the user's current condition/state may then be utilized for actions such as adjusting parameters on the user's dashboard, engage the user in various activities, display alerts that are physical to the senses such as for example visual (dashboard changes, flashing lights), sounds (alarm, alert), and tactile sensations such as vibration or movement of a user device. In one embodiment, all such actions may be implemented at the same time on a single user device (e.g., user's cell phone).

In another embodiment, the various data-points (vitals, GSR, etc.) may be collected at or near a set of locations (e.g., any of the locations where a user travels or visits over time). Those data points then each accrue an average, a baseline, standard deviations, and/or other statistical values at each location which may then be used to evaluate a user's current condition by comparing to the user's current data-point value(s) at one or more of those locations.

In another embodiment, a plurality of different user devices (e.g., mobile devices, automobile, IoT devices, etc.) each provide one or more physical actions and that one or more may require a response form the user to turn it off. In one embodiment, nearby IoT devices are operated in a manner that requests user response or serves to notify the user that a current state/condition needs to be addressed (such operation may include automatically requisitioning such devices and permissions to do so may be set, for example, in a user's profile). In one embodiment, nearby light sources are flashed in an annoying manner to distract he user or otherwise prompt action which the user will take (e.g., such as a self-assessment) or other recommended action completion of which will return the light(s) to normal operation as before the alert.

In one embodiment, a multi-dimensional graph is output on the user's (or provider's) screen to visually show the user's states over time in relation to the displayed interrelated indication(s). The display may morph over time as yet another indication progresses. In one embodiments, the indications used as a basis for the 3-D graph and it's morph(s) are the most relevant (or most identifiable to the user) factors in the user's current condition and the display provides the user with a practical user friendly visualization that helps the user understand and realize the impact those factors have on his/her well-being (accordingly, a display for one user may be based on three entirely different indications than for another user). To the physician, therapist, or other professional, it provides a talking point to discuss what is going on to cause the user's current condition as well as a representation of the user's condition. In one embodiment, the most relevant factors to the user and to the physician/therapist are different and different graphs may be produced and displayed to the various recipients.

Prior systems do not factor in the user's current location or proximity in relation to other indications, and various embodiments as described herein take advantage to the user's mobile device for gathering and monitoring proximity information (in one embodiment the user's location in proximity to another device's location is considered, such as the user being close to an automobile's location which may be a factor used to elevate the user's risk or trigger an alert). Such proximity information may be the immediate information of the user's location and collection of location information over time to establish a progression of events and/or timeline which may be evaluated against historical user locations and like-situated individuals (e.g., the progression of the user over time through locations considered to be elevated risk and then, at 2 AM, a location matching the location of the user's automobile may trigger an alert which may include a disabling signal to the automobile). In one embodiment, a combination of emotional state and BAC disables the automobile. In another embodiment a combination of emotional state, vitals, and BAC disables the automobile, and the combination may, for example, be based on a number of points awarded to each of the emotional state, vitals, and BAC such that a total number of points higher than a threshold regardless of which of the disparate components provide the points is determinative in whether the vehicle is disabled. The disablement may be performed through an engine control module that may also control lights and other features of the automobile (e.g., flash lights if a user attempts to start the car).

The various embodiments also take advantage of the attachment most people have to their mobile devices such that the system is not easily turned off or left out of the user's current situation. This is an advantage not recognized in the prior art systems especially when considering a comprehensive analysis of other factors/combinations such as those described above and their correspondence to past activities that can then be used to establish the user's current level of risk as described herein.

Perhaps more importantly, it should be understood upon review of the disclosure as a whole that the steps or processes described (and ultimately claimed below) are a genus of rules that do not preempt all techniques for automating relapse risk that rely on rules. The various constructs, rules, and processes described herein are rendered in a specific way (sequence, timing, and weight) such that broad preemption is not possible and in fact prevented (e.g., relapse prevention does not require any of these rules).

The app may recognize an in-person conversation via an open mike with a voice identifier running, or the app may ask the user to identify a conversation during or after it is occurring (which may then be used to recognize subsequent conversations with the same person). If such interactions are consistently negative (e.g., as mapped against GSR or other biometrics) appropriate counseling for the individuals and/or pair may be suggested by the app, for example, suggested to the team leader who may then discuss with the user.

Other biometric data may be similarly utilized and correlated with other events. Types of biometric data such as breathing rate, muscle activity, brain activity, activity of different portions of the brain, or any type of physical or neurological metric, from any type of sensor (e.g., muscle sensor, EEG sensor, etc.) that may capture that activity/metric may be similarly utilized as discussed herein with respect to GSR or any other biometric.

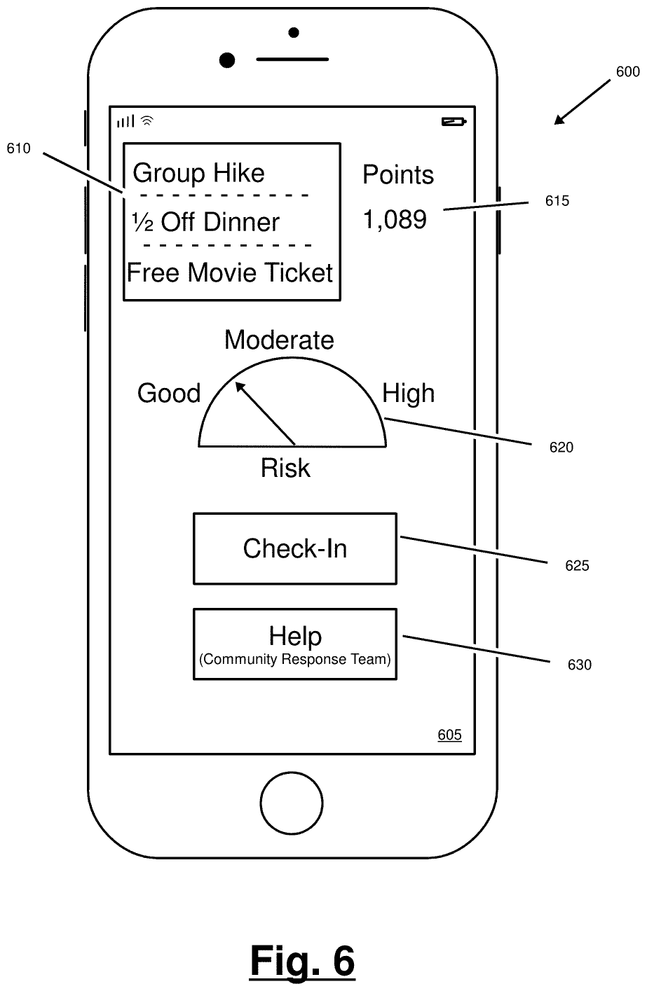

FIG. 6 is an illustration of a user interface 605 according to an embodiment. A user's mobile device 600 hosts an app that displays the user interface 605. The interface hosts messaging summaries which may be, for example, event notifications (e.g., group hike) and specials (e.g., dinner discounts, movie tickets, notation of point awards, etc.). Such specials may be, for example, awards for reaching certain milestones, gifts from groups supporting recovery, etc. In one embodiment, such notifications appear through the mobile device's messaging system as set-up in the mobile device's preferences.

A points area 615 displays the user's current points total, which may highlight recent awards. Such awards may be granted from a pool of award points for successful completion of milestones, check-ins, maintaining a diary, etc. A risk meter 620 may provide, for example, an indication of a user's current risk profile, an indication of the user's current emotional level or mode or mental condition, or other evaluable condition or state. In the case of a risk profile, the app or system (e.g., Back-End 142) accesses the various conditions and trends currently affecting the user to produce the risk profile—this provides the user with a feedback mechanism from which can help the user judge their own current condition and add an extra metric to any decisions about activities. In one embodiment, tapping the risk meter (or another icon, drop down menu, etc.) brings up data or a mini analysis that may show, for example, a set of data points (e.g., most influential data points) from which the risk factor was determined. For example, data points from the relational database, trend analysis, etc. providing additional feedback, information, and explanation to the user or their respective sobriety network.

Check-In button 625 may be utilized to respond to an app generated request for a checking, or for a user-initiated check-in. A help button 630 may be used when the user feels conditions warrant an immediate reach out to his community (e.g., 911 to user's support community). The button may initiate, for example, a call, video conference, etc. with one or more team members who will interact with the user to discuss the current situation and provide a plan to address any difficulties. In one embodiment, at the same time, a response team may be notified to make a physical intervention if warranted. A warranted intervention may be determined by team members on the call, or other protocol (which may be automated based on circumstances such reported location, risk factors, contract with user, etc., for example). Team members on a call may communicate via a specialized interface that allows them to, for example, suggest an intervention by pressing a button which then automatically asks the team leader (and/or other team members) (e.g., via notification on the specialized interface) if they would concur or support intervention.

Such suggested intervention may, for example, be privately shared between the suggesting team member and the team leader or with the entire response team (e.g., pop-up notification on team member device: “Team member George suggests an intervention”). The suggested intervention may be considered privately or discussed on the call at the team leader's discretion (e.g., depending on circumstances).

The response team may be initiated by an automated message to one or more team members (not necessarily on the call) with, for example, particulars such as location of the user, brief/recent history, and a link to the video call feed. The response team may be composed via automated computer selection based on, for example, from a pool of available team members in order of, for example, experience, proximity, current situational knowledge, etc. Substitutes may be automatically selected, notified, and verified in response to first round selected team members who are either unavailable or non-responsive.

A user interface according to an embodiment may provide facilities for, for example, data feeds and communications with processing for maintaining event logs, access to self-help, preferences, social network feeds and links. Separately or additionally, schedules may be applied, for example, to guide the individual through a routine or daily activity and provide easy check off and suggestions to modify or organize behavior, for example, to reach a next task completion or milestone. Such task may be, for example, completing work, grocery shopping, and/or going directly home.

Such tasking may include a shopping list for a suggested meal and recipe for the evening's meal (or a suggested take-out, which may be, for example, guided along a preapproved drug/alcohol free route). Such tasks may be linked to social media posts as required by the program (e.g., on Facebook or local recovery page). Posts made are also data points recorded and used to analyze current and future activities/conditions. Such task may be general in natures or provide specific instructions or even micro-management of the user's activities (which may depend, for example, on the user's experience, fluidity, or level of acceptance of the program/app). For example, a user's trip across town may be provided with a specific route and specific way points, and, missing a scheduled waypoint or destination time frame or route may result in a notification to team members.

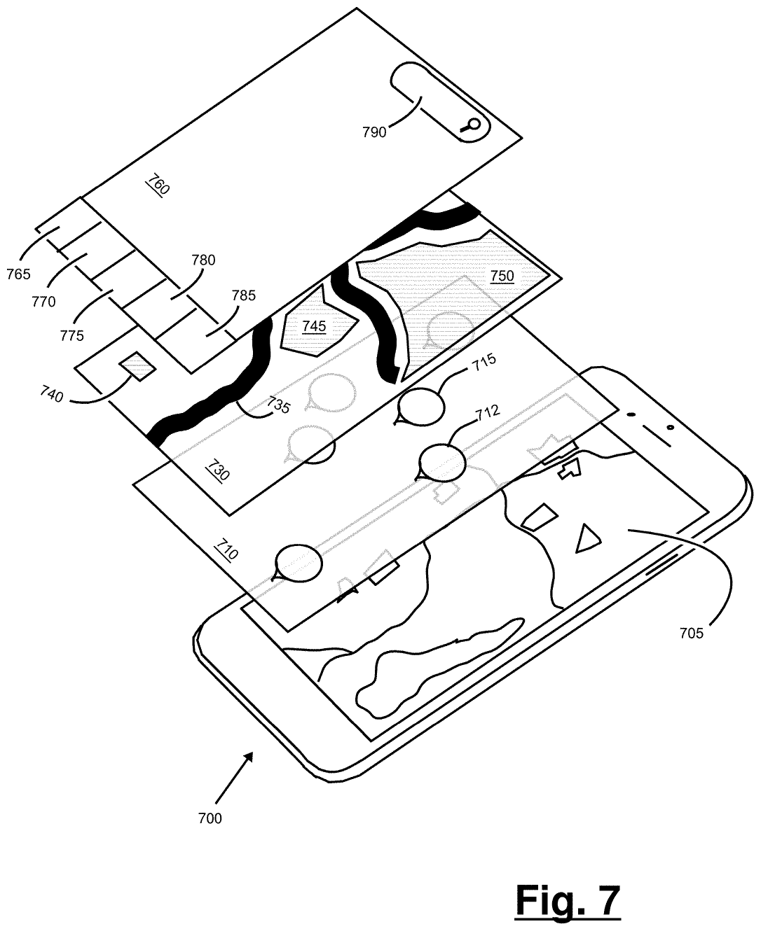

FIG. 7 is an illustration of a user interface, map, and other features according to an embodiment, on, for example, a user's mobile device 700. A map 705 provides a base layer of the user's surroundings. Overlay 710 may include locations of friends, contacts, community support points, places of solace or meaning to the user may be provided with markers (e.g., markers 712/715, for example). Such markers may be customized by the team leader or licensed therapist, especially initially, and controlled until early or other stages of recovery having higher relapse potential are past.

Such markers may be vetted via AI/machine learning routines to ascertain an appropriateness of the marker. For example, certified or trained support members would likely pass vetting, contacts may pass subject to additional vetting designed to weed out potential undesirable influences such as those that might be enablers or not fully in-tune to the user's situation. Liquor stores would most likely be excluded, as would locations of previous re-lapse or breakdown events, for example.

Such markers may include any of icons, photos, graphics, or text associated the person/contact being marked. Such markers may, for example, be touch sensitive to provide immediate team or support structure communication. Overlay 730 may include geo-fences or outlines of forbidden zones or off-limit areas that allow the user to plan a route that does not include things such as, for example, known bar neighborhoods, areas of problems for the user, known drug alleys, etc. For example, overlay 730 includes OK route 735 and off-limit areas 750, 745, and 740. Such forbidden zones may be determined based on known neighborhood characteristics (e.g., an area known for nightlife/bars), known previous issues with the user (e.g., location of a previous relapse event), or data collected with respect to the user that was negative whether or not the cause was determined (e.g., an area that consistently causes a high GSR response when passing by).

Top overlay 760 includes access to application (app) features such as search 790 (e.g., search for support or community members/resources), Location 765 (e.g., centering map on the user's location), List 770 (e.g., populates the map with markers representing the user's support team/facilities, or the user's customizable list of people or locations, for example), Favorites 775 (e.g., user's favorite or memorable locations on the map, for example, or, links to other platforms, web browser, social media, etc.—any of which may be monitored or used for data collection for other functions of the app, for example), Circles 780 (e.g., selectable sets of various contacts, support group, work group, clubs, etc.), and profile 785 (e.g., set up the user's profile that may be accessed by friends, contacts, team members, etc., the same profile may be utilized, for example, to populate profiles on other apps such as Facebook, twitter or others, and again, may also be subject to monitoring/data gathering for other application functions, and such data gathering may be by agreement (e.g., opt-in agreement) with the user, court order, and/or permissions granted for the user's various accounts, for example).

Accordingly, various embodiments may be very sophisticated and a user's app may be set-up by a rehab facility according to conditions of re-hap, court-order, or under a general program (e.g., community or group funded or via insurance) which may include cash or point awards. Such programs may include pre-arranged persons of responsibility such as team organizers, response team members, etc. Such team and/or response members may be part of the user's existing sober network or other stakeholders in the user's recovery. Alternatively, such team members may be composed of paid professionals who would then authorize or advise on bringing in the user's existing network on a case-by-case basis. In yet another embodiment, no network, no insurance, and/or no rehab is utilized or available and the user may download the app on their own and use it as-is without additional outside help. In yet another embodiment, the user may opt for paid services through the app, for example, when at a high risk or low point emotionally, the user reach out by (or app may suggest) spending $50 for a counseling session with counselors that may be contracted by the application.

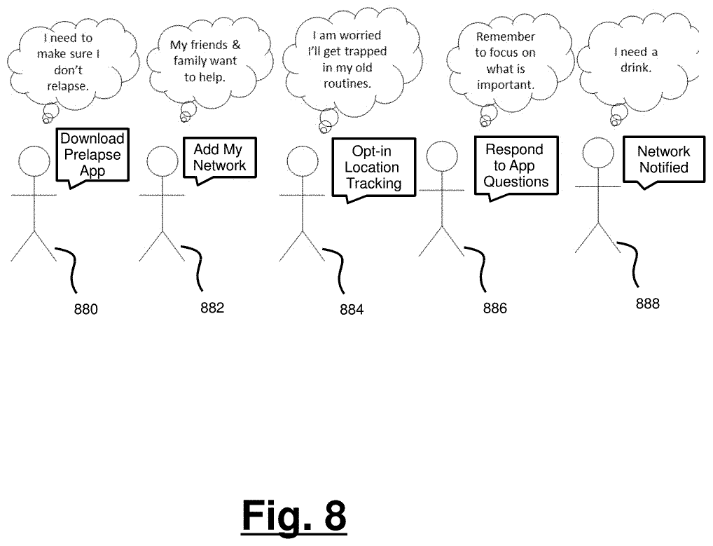

FIG. 8 is an illustrative flow of a scenario according to an embodiment. An individual user or patient download's the app (application) to his/her phone, personal device, or laptop, for example (step 880). Any internet appliance may be utilized, and, depending on the individual's personal situation, such download and operations discussed herein may be performed by a handler, secretary, personal assistant, etc., however, please note that some embodiments may include questions, games, quizzes, or other activities that are designed to be part of an overall recovery and treatment and should, for best effect, be performed by the individual).

Once downloaded, the app may prompt the user for relevant information, which may include, for example, answering a list of short multiple-choice questions. The questions may be designed to evaluate the individual. The evaluation may be, for example, to establish an emotional baseline. The evaluation may be, for example, to determine if a crisis or other emergency situation exists right now, at this moment (which may have been why the individual downloaded the app at this time, for example). In one embodiment, the app can determine its own baseline over time through use of the app. Even if just left running in the background on the user's device—a baseline can be established through collections of events and activities—though less timely than if the individual establishes it from the beginning. Time is of the essence as preventing relapse because it is a most dangerous event for the individual, stakeholders, and non-associated individuals that may just happen to be nearby, so the user may be pressed on this point, and in one embodiment, failure to establish a base line does result in a personal call or contact to explain the reasoning and why it is important.

A next step establishes the individual's network, health providers, etc. For example, the app may direct the individual to input his/her sobriety or support network (e.g., step 882). This could include but is not limited to Parents, sponsors, therapists, life coaches support groups etc. Participants in the end-users sobriety and/or ongoing mental health support. The input may include phone numbers, social media handles, etc. In one embodiment, well established networks may include drop down menus for selection to national organizations, or local chapters in the individual's immediate geographic area. In one embodiment, past location data, social media posts, contacts, etc., may be analyzed for clues as to associations and established networks and such choices may be pre-filled or suggested by the app.

Once running (even if not registered or fully established as to baselines, networks or personal information) the app begins to gather data from various sources (e.g., opt-in for location tracking, step 884). For example, throughout the day, the system (app) may interact with the end-user. Such interaction may, for example, be based on the answers to the questions while setting the app up. In one embodiment, such interaction may include asking the same questions again or asking if the individual is feeling differently about any one or more of the questions. The app may, for example, ask the individual to report on their respective emotional health multiple times per day (e.g., step 886). The frequency may, for example, be determined by the answers during setup (e.g., an evaluation of the user's current level of recovery).

The emotional health may be captured multiple-ways, including, for example, through a personal device (smartphone), e.g., the user's mobile device on which the app is hosted (or via logon to a website, etc.). In one embodiment, the emotional query may take the form of a pop-up prompt, text message, or alert/notification with a response capability (e.g., radio button selections, link, etc.). For example, a user may be prompted to select one of multiple possible answers, such as requesting the user press one of any number (e.g., 7) of choices about their current emotional state (e.g., from Amazing to Poor. and one or more, even several options in between etc.). Such queries may occur multiple times per day (e.g., 7-15× per day), or at predefined junctures (morning, arriving at work, lunch, break time, etc.).

While capturing emotional state remains an important priority of various embodiments of the app, the frequency of direct user-app interaction in capturing emotional state may be varied depending on the stage of the user's recovery. For example, a sliding scale of a mix of direct questioning/queries and automated data collection transitioning to more automated collection as the user progresses in recovery. Such a transition may be further facilitated as the app learns or becomes more familiar with the user's habits and activities compared to established emotion states in similar circumstances.

Such queries may include actual actions over answering question, such as requests to send a selfie or face shot. Such photos may be logged and entered into facial or emotional recognition system supported by, for example AI for analysis of the individual's emotional state. The photos may be correlated to past events and past photos to recognize facial trends leading up to highly charged emotional states and/or potential relapse or other events. In one embodiment, social media images are analyzed for similar purposes, and the system may also commandeer the user's mobile device camera (with privacy permissions appropriately set, for example) to evaluate facial expressions (e.g., FEA), features, etc., at other times as well.

In other embodiments, other interactions of various calibers may be utilized or recognized, and such other interactions are not necessarily direct. For example, the app may collect information from other apps from which it has appropriate permission. Such collected information may be, for example, a user's record of wins/losses in an online game, or an amount of playing time, for example. It may be inferred upon experience or other factors what the significance of any such interaction may be with respect to the user's emotional state/health, but presumably, successful completion of certain games may be viewed positively and provide some data for a complete picture of emotional health and leaving a game incomplete (particularly if a history shows completion combined with good emotional status). The same data may be combined with other events, which if negative, (particularly if the game playing is seen as negative, as in on-line addiction, for example) may point to a less stable or more difficult emotional health situation.

Accordingly, the app may collect data by direct inquiry, background activities (locations, purchases, etc.), and through, for example, use of other apps or facilities. In one embodiment, direct inquiry may be the primary (or single) source of emotional data. In other embodiments, a variety of sources are tied together to produce an emotional score on which other decisions (e.g., assigning action items) may be based or modulated. The various embodiments may include or be described, for example, as collecting data in the background and preparing information supporting overall structure/habits of the individual, and such structure/habits may provide a baseline from which to compare new events affecting the individual's emotional status.

For example, the app may monitor the end-user/individual's typical days from the time they get up, to what they do daily and where they do it. Such activities/locations may be reported/recorded in a comprehensive manner. Consistency within such activities/locations may be rewarded. The app may, for example, keep a running score of points or a percentage value reflecting how well the individual is able to stick with a consistent program. Such score may be reported to the individual through pop-ups or via a main page of the app. The current score may be shown on an icon representing the app on the user's mobile device home page. A good score or high percentage indicates the user has been staying on track, has consistency, and/or is essentially running a good program/life and/or exhibiting, for example, good personal accountability. Such scores may be reported, for example, to stakeholders, insurance companies, etc. Progress or good scores may be re-numerated with gifts, coupons (e.g., “Your local Starbucks says congratulations—coffee on us!”), or cash (even small amounts) from stakeholders, insurance companies, or community organizations for example.

Rewards in the form of virtual badges, trophy's or other status may be supplemented with a physical letter or shout out at a local meeting (e.g., meeting leader notified via text/notification for example). Charts may be developed and shared with a user to show progress over a week, month, or other time frame—further reinforcing progress/status. The app may include facilities to receive appeals or requests for further points based on circumstances or specific challenges which the individual has successfully navigated.

Inconsistency of normal habits or routines or other indications may point to a relapse event. Other indications may potentially include an outright admission by the user (e.g., “I need a drink”) which may be an answer the user gives to one of the periodic questions, or a statement captured on an open mic. In such cases, the user's network is notified, or the app takes other action to help move the user past the current situation (e.g., telling the user to change his location immediately, or check-in to a specific sobriety group, etc.).

Data collected may be correlated to other data and to deeper patterns that, while may be applicable to the broader class of individuals or even applicable to all users, but most applicable to the individual user may be identified by the app and shared with the individual, his/her counselors, medical professionals, etc. Such deeper patterns may be, for example, a realization (or identification by the app) that going out to certain activities (e.g., staying up too late) throw off an individual and puts them in relapse danger. Such identification of areas where emotional deviations occur will identify much more detail about the real problem(s) the individual is facing (and potentially other user's as well)—thus reaching to the core, deeper issues that root many Drug and Alcohol problems (i.e., the drug/alcohol problem being the symptom). Accordingly, the app (and an overall premise that may be applied) collects habits, movements, and other data about the user and activities, events, etc., along with their personal emotional state, including that emotion state as the user feels it/as they understand it/as they respond to it. The identifier patterns, triggers or other information is then applied to decisions about future events, notifications of stakeholders, response teams, etc.

In operation, various features or embodiments will play-out depending on activities and circumstances as they develop relative to the individual. For example, tracking movements of an individual and identifying potential issues with an establishment or part of an establishment visited. An evaluation may be performed to determine a level of response, which may be, for example, a text or alert asking the individual are you on-track for this evenings goals—any issues please call,” for example. As the day progresses a more serious event r circumstance may arise which may elevate the response to a direct call, or the app taking control of the individuals mobile device to start streaming audio and/or video to a help center who may then quickly decide if the situation is elevate to a full intervention or other means to alter the individual's current course. Such events may include, for example, detection of suspicious patterns around a bar or known drug source neighborhood and feedback from the individual's mobile device (or individual's automobile's on board system) indicating driving.

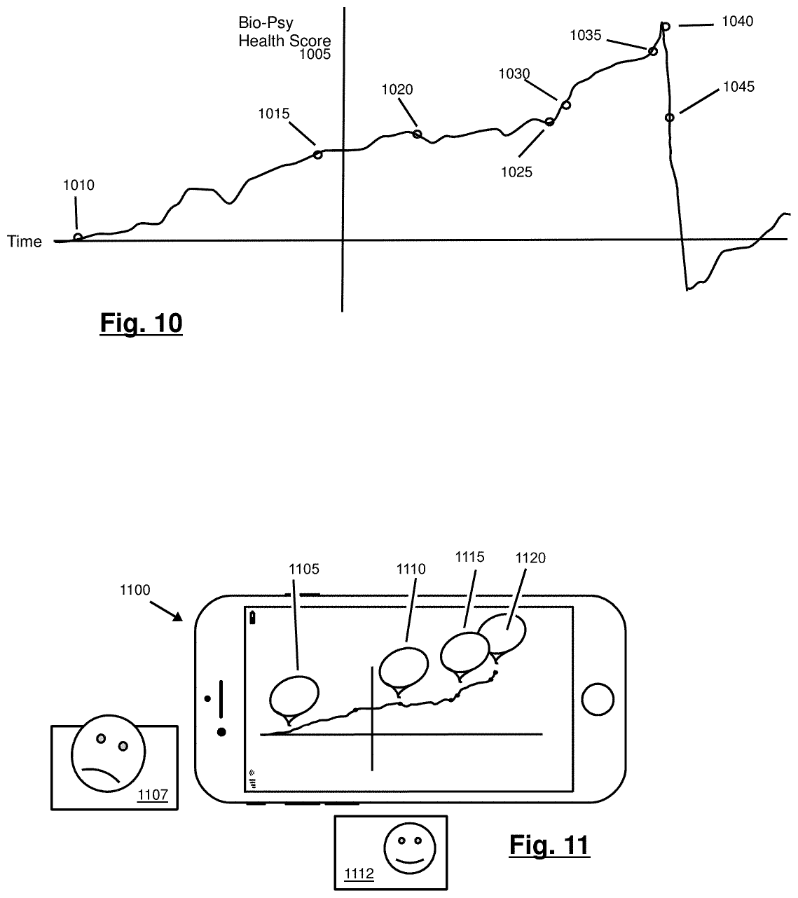

Further various trends may be realized such as increases in emotional state which may raise alerts especially if rising rapidly. For example, FIG. 10 is graph illustrating a user's emotional state according to an embodiment. The bio-psy health score 1005 may be, for example, an score determined from one or more factors including any of the data points discussed herein and may be averaged over an interval such as a 24-hour period. For example, GSR, heart rate, EEG (e.g. one or more of the various brain wave frequency bands) or a combination (e.g., GSR and vitals, etc.). What the graph may show, for example, a user at a relatively low point 1010 which may be while the user is in rehab, and increase in the score as the user prepares to exit rehab (e.g., 1015), and then various ups and downs all within a reasonable range (e.g., 1020, 1025, and 1030), but then experiencing a sharp increase out-of-range and at a high rate (e.g., from 1025 to 1035 and then peaking at 1040). All of this typically occurring without a user's conscious thought about the situation. Among many important aspects is the ability to track the score and recognize sharp increases at an unusual rate or reaching unusual values (e.g., outside a standard deviation of values or rates of increase/decrease), which may trigger an alert or one or more physical actions, such as messages or inquiries to the user or team members. The graph goes on to illustrate a peak 1040 and subsequent crash 1045 and on which is a relapse event to be avoided.

In one embodiment, one or more of the data points may be a combined amount of activity such as brain wave activity (e.g., recorded via brain wave sensors) over a variety of brain wave frequency bands (e.g., any combination of Alpha Beta, Gamma, Theta, or other bands for example). The frequency bands may be customized to the individual, for example, in the case where a user typically shows activity in key frequencies between two established frequency bands, a specialized band may be monitored for that individual (in one embodiment, a specialized detector is configured to exclusively monitor a previously identified “custom” brain wave band for a user which has been show to provide critical activity at important emotions states or junctures for this user). In another embodiment, the frequency bands are monitored individually and provide separate data point. In yet another embodiment, the frequencies and location of the brain wave activity is recorded, which may be determined from placement of multiple external sensors or, in extreme cases, internally placed very low power sensors in communication a relay device. Over time, it can be determined if any particular user is predisposed to certain brain wave combinations and/or locations indicative of one or more relapse scores, such as, for example, well adjusted, about to crash, or engaged in a full relapse event (e.g., drinking or substance use, for example). Similarly, they can be attributed to various emotional states that may be determined, for example, by comparing activity in close proximity to a user self-assessment score.

In another embodiment, a user is initially equipped with highly sensitive sensors at many locations around the skull (e.g., in a head-gear like sensor array) such that highly accurate brain wave information is recorded. The user uses the headgear for an initial period of time and carefully records emotional states along with the location, GSR, vitals, and other data points. Over time, an accurate picture of the brain wave data points and particularly their combination with the other less intrusive sensors is developed. For example, the brain wave sensors will be seen as interrelated with GSR more or less for certain types of brain waves, and linked to one or more emotional states. In this manner, after the initial period is over, the user no longer needs to wear the headgear as the other sensors can accurately predict the brain wave content and more particularly the emotional states associated therewith.

In yet another embodiment, as a user reviews a timeline or graph of emotional scores (e.g., as described in FIG. 11, for example) and the user looks back, the user may, for example, click on one of the bubble points and confirm a level of emotional content for that day/event which may be cross checked against stored brain wave activity (frequencies) for the same time period. Such review/looking back analysis may then refine the values associated with the brain wave activity and the graphs may be adjusted accordingly. A similar review and adjustment of the other data points may also be performed. And again, like the other data points collected, the brain wave activity may be cross-referenced/correlated to location and/or other data points. Accordingly, the brain wave activity may be weighted as a whole or any of its individual components according to location or other data points as may also be performed for GSR and location or other cross references/correlations that are described herein.

FIG. 11 is an output 1100 provided to a user to show the user emotional conditions over time according to an embodiment. The various embodiments describe other items that may be shown to the user to help the user recognize the emotional states s/he experiences, Here, the aforementioned Bio-Psy score and graph is shown to the user at a time (e.g., 1115/1120) when the increases in the score are outside normal daily fluctuations. The user may look back over the past days, week, or month and correlate how the user was feeling then to the graph and upon reflection of the activities or events in which the user partook, thus helping the user see how the trend evolved and help the user get a firm grip on the current situation (whether it be positive or negative), and hopefully before a crash or degradation occurs (such as shown previously at 1045). Further, graphics including icons, characters, or snippets from user selfies or pictures from events (e.g., images 1107 and 1112) may be places in the bubbles at each of the junctures (e.g. 1105, 1110, 1115, and 1120) which can help the user more quickly realize that factors are influencing, causing, or are a result of the user's emotional condition and again helping the user recognize what is happening so that it may be more effectively addressed.

The Bio-Psy score may be, for example, a combination of readings from the various modalities and instrumentalities available from the smart phone or any monitoring devices attached or utilized. The values may, for example, be normalized added or averaged. The various scores may be weighted based on their apparent influence in previous emotional states, recovery, or crashes. Alternatively, the Bio-Psy score may be a reading from a single modality, such as GSR (e.g., normalized for the graph to be displayed). And, any such readings may themselves be normalized as to the user's location at the time of the reading.

The displayed graph may further be superimposed with other graphs or include back and forward arrows to allow the user to view and/or flip through several different graphs reflecting his/her emotional state over time (e.g., a GSR graph, a blood pressure graph, an eye movement graph, etc.). In one embodiment, a plurality of graphs (e.g., GSR, self-assessment, and vitals) are normalized for the same coordinate system and superimposed, the graph most effective may be in bold and the pother graphs greyed out in the background, and the use back/forward buttons may allow the user to exchange the next graph to be bold and the previously bold graph now greyed-out in the background.

Further, such graphs may be selected for the individual user based on the factors that have or appear to have the greatest influence on the user's current condition, which may also change or be adjusted as the user continues to use the platform. For example, a user whose graphs mostly rely on GSR, but circumstances over time shift and it is recognized that heart rate or another vital appears to be having changes consistently with a user's reported changes in emotional state, then heart rate may be a primary chart (or even replace GSR) to show a user for evaluation when emotional conditions change. Similar adjustments may be made to a combined Bio-Psy score changing weightings and even removing, replacing, or adding new data-points.

The application or other software may be running on the mobile device or hosted remotely (e.g., accessed via an API, subscription, or pay service call). National statistics, alcohol and chemical dependency best practices, geospatial technology and the ability to capture individual end-user data may be built into the application or accessed via local or remote storage and such data sets may be utilized in comparing the individual's data and/or guide decisions on a level of intervention or support to be issued.

For example, these data sets, coupled with a collection of real-time emotional cues, may be used to arm recovering individuals with a personalized app experience. Thus, the app may identify specific problematic relapse triggers based on individual data by detecting potential relapse cues and triggers prior to them becoming a full relapse episode. The same may be compared or evaluated against national statistics and the individuals known prior history.

In various embodiments, the application is customizable by the end-user and can be accessed by any personal device. Such access may be over the Internet or be an installed Android/iOS application. By collecting and processing a myriad of emotional, psychological, geographical, and social matrix, end users are in a unique position to monitor, track, and react to emotional deviations that can lead to relapse. Again, such collection may include seemingly unrelated activities and can be correlated back to events and used for projections under similar or other circumstances.

In addition, the app may be configured to alert multiple participants who are stakeholders in the end-users recovery process. Stakeholders are participants in the end-users “recovery network”. Because the app is highly customizable, various participants within the end-users recovery network can be notified based on a number of criteria, including but not limited to, the severity of relapse danger, relapse location, and their relationship to the end-user. The app is a companion for recovery support, fits perfectly (fits well, for example), and can aid in alcohol and drug-related support groups. The app may include capabilities in support of the above, which may include, for example and one or more of:
Take control of phone to send images to counselor
Take control of phone to get immediate questions answered (therapist calls, Mr. X, you stopped near a liquor store, let's talk.”)
Real-time credit card information. Purchases.
Link to liquor drug sales outlets
GPS coordinated map of all liquor/tobacco/cannabis/etc. sales. Areas of town known for drug sales. Locations of beer in grocery stores.
Geofencing—notifications, alerts, not allowed to pass, etc.
Take over phone to get images/audio of activities.
Signed waiver of privacy—user selects acceptable level.
Warn user before entering a liquor zone. Call from app, call from autonomous voice, call from stakeholder, Group call e.g., stakeholder, therapist, AAA co-member. May be based, for example, on severity of event.
Differentiate from pre-cursor and full-on trigger events.
Different customized response, from autonomous virtual assistant to full on intervention.
Full on intervention with QRF—rapid response team on the way to location.
Full on virtual intervention group chat call when needed.
Provision of automated reports to providers, stakeholders, individuals/patients, insurance companies, etc.
Billing to insurance companies based on performance (e.g., collecting bonuses for miles stones, days sober, etc.).
Managing groups of interveners, paid response forces for high end implementations and/or volunteers, and volunteer forces trained or guided for community based service organizations, for example.
Dispatching an intervention APB (All Points Bulletin) that may include, for example, last known location, pictures, bio, known circumstances, and predicted current issue. An APB alerts responding forces as to potential issues, background etc. and. For example a suggested intervention style. The application may organize the intervention along proper guidelines (e.g., making sure two people are present and available and confirming, tracking their locations, turning all cell phone cameras on, for example—many variations on the protocols may be implemented.)

The significant amounts of data collected (and utilized) by the app and may be used for reports or other paperwork relevant to a user's rehab, insurance, etc. Such data is extensive and is able to provide insight in to the user's overall well-being and may be analyzed or reviewed to find patterns or underlying issues which may be the root cause of the dependency issues to begin with. Such analysis may be performed by an analysis module that searches for activity/emotional patterns related to one or more relapse or low emotional state events in the user's data history and the results may be provided to counselors or other authorized health professionals (or directly to the user) depending on permission levels set in the user's preferences, for example.

In one embodiment, likelihood of relapse is systematically determined by specifically providing a combined platform supporting an application on a smart phone that coordinates a plurality end data points (such as indications) to predict meaningful outcomes in physical response including obtaining a first set of rules that together define output stream of data representing a likelihood of relapse. Relapse is a process which may have one or more events. As a result, there are numerous end data points that can successfully predict the onset of relapse conditions. As a function of time place, emotional state, geo-spatial location, biosensor indicators, mobile analysis, facial recognition, and gait analysis and other related technology. Multiple AI and ML computations ran, (i.e. users past behavioral fingerprint/habits in concert with past habits and subsequent actions of similarly situated individuals with in-kind datasets. Obtaining a data file of current user conditions comprising a plurality of sub-conditions conditions comprising at least one of events, emotional levels, and mobile analysis. Bio-sensor data, facial recognition outcomes, gait analysis, and geospatial locations related to a user's current situation and recent activities. Generating an intermediate stream of output data weighted based on a similarity of current and recent events to the output stream of data and further weighted to the extent the output stream of data represents the user's past data by evaluating said plurality of sub-sequences against said first set of rules. Generating a final determination of likelihood of relapse based on the weighted intermediate stream of output data and selecting a presentation format and actions for the user's smart phone and auxiliary resources to address the likely relapse. And applying the final determination in the preselected format on, providing a pop display informing the user of conditions currently being experienced and having the user acknowledge the same, instructing the user to proceed in a particular direction or on a particular route (i.e. generating messages supporting continued self-care and ongoing recovery); and in critical conditions launching a full intervention. Applying the auxiliary resources by sending alert messages to clinical team members, therapists, phycologist, fellowship/support group, family and healthy pear support, with various data sets on the user's condition, location, including an interactive map showing a current location and course of the user, and coordinating communication with the user while enroute to intervene. The pre-selected format may be, for example, a 3-dimensional graph showing one or more of the indications (e.g., data end points) over time, or a meter, or a score, for example.

Preexisting recovery/prevention approaches are tedious and time consuming, generally requiring appointments, time in rehab facilities, as well as being inaccurate due to the large number of subjective analysis required. The present embodiments overcome many of the deficiencies of the prior art (but does not supplant them entirely) and obtains its objectives by providing an integrated method embodied in computer software for use with a computer for the rapid and consistent determination of a user's current state/risk of relapse and provides efficient manners and different levels to address the same consistent with the user's current state in a very cost effective manner. Accordingly, it is an object of various embodiments to provide a method for automatically producing accurate and continuously improved risk assessment in the case of the AUD/SUD user. In various embodiments, automation of tasks needed for assessment of a user's relapse condition is provided and may be determined without human intervention or subjective analysis (which is not excluded or may be excluded entirely) including emotional state analysis, consideration of vitals, correlating any one or more to physical factors such as location, time-of-day, week, or month, recent history, or history as a group, for example. This automation may be accomplished through rules that are applied to the timed occurrence of real events and applied weights of those events. Importantly, the timing of such information may also be utilized to provide a better picture of condition, and such information and its timing or sequence of events are compared and analyzed according to the individual user's history of such events (e.g., events and biometric data) and sequences. The embodiments go beyond (that are different from) simply determining use of substances and physician/therapist evaluation of the user's state. Instead, these rules include the production a more empirically determined evaluation of the user's state or current risk based on the context in which the user is currently in and updates the same as new contexts occur.

One exemplary set of rules provided and applied in this specification is taking multiple indications together and determining their relevant (weight) based on their contributions to, for example, past relate events or risks. This exemplary set of rules provides for specific actions to address the user's current risk through varying levels of intervention, including the visual display which allows the user to recognize that condition and allow the user to come to grips with his/her current state to help allow the user to make a conscious decision to address the same which may be with, for example, one or more suggestions from the app/platform. Such suggestions can be a simple game or discussion of the user's state (e.g., with the help of the visual aid), or something more immediate and physically consequential such as a full-on intervention or a disabling of the user's automobile, for example—all of which will vary depending on the circumstances, assessed risk, and the user's history. That is, the rule automates both a response without human intervention and may be comingled with human actions expressly directed by the platform.

As a whole, the embodiments (and various claims provided herein) are not directed to an abstract idea because they generate a tangible product, namely an empirical analysis and display of a results for visualization by the AUD/SUD user, and direct and/or monitor affirmative actions directed at addressing the current situation (e.g., monitoring team member progress and weaving that progress into interactions with the user to help make any intervention more successful) and such direction may include launching a drone or other automated device to assist in a full or partial intervention, monitoring, or physical action with respect to the user, and may include directing IoT devices to behave or operate in a manner that is helpful for such intervention (e.g., engaging an IoT device to send a message to a programmable display billboard congratulating the user on a certain anniversary or number of days of sobriety when the user is in position to see the message). The various embodiments are technological because it provides for automated and automatic analysis of a user's condition without human intervention that can be updated near continuously without a physician, therapist, or other team member's constant intermediation, and includes responses as well that do not require their involvement (and, although, such involvement may be elicited in other embodiments).

The various embodiments (and any claims derived therefrom) effect an improvement in the technology or relevant technical fields, specifically the determination of a user's current relapse condition or risk and projection into the future based on the user's current state and path leading to the current state and in consideration of the user's past states and corresponding conditions. This improvement, results from the embodiments that employ specific rules and specific types of rules and uses those rules in a specific technological way as described above and elsewhere herein. The claimed type of rules are, for example, as they are limited by the claims, rules that define weights for the various end-points or indications that together are combined and may be further analyzed of weighted based on historic information, including a sequence in which the indications and/or physical data points are realized. When applied, these rules adjust for the user's specific circumstances and the users past conditions/circumstances and determine the most likely best actions to be taken under the circumstances.

These limitations are specific because the rules will necessarily vary by individual user, for example, a person who in the past only drinks at bars or other establishments such as in group setting may be entirely different, have different indications, different weights, and different sequences of events that typically increase relapse risk and/or progression. Further, the embodiments as recited in the claims cannot preempt the field because other techniques exist that provide similar information and make determinations as a user's emotional condition and for the treatment of AUD and SUD—they do not however, provide the same evaluations and fail to teach or suggest the embodiments as claimed.

The embodiments, and specifically the claims “use techniques together in a process designed to solve a technological problem in ‘conventional industry practice,’ that problem being supporting an addict's recovery whether AUD or SUD related. The invention itself goes further by providing the best chance for stopping a relapse event before it happens. The invention uses a multi-faceted analysis (e.g., images, mood, location, history, and other indications data/end-points). Accordingly, the embodiments as claimed improve an existing technological process in a wide field of treatment and are not merely provided as being implemented on a computer or smart phone. That being said, the smart phone is a device most suitable for such implementation because it is portable, always nearby, and may be outfitted with all the range of technologies (specific combination of technologies) used to implement various disclosed processes.

The improvements here include allowing computers to produce meaningful event notifications and suggestions to maintain ongoing and healthy recovery that previously could only be provided in a labor intensive rehabilitation environment. In fact, many aspects of the embodiments improve the overall rehabilitation environment, and, in one embodiment, the platform and smart phone are introduced to the recovering addict while in a rehab facility where not only the platform begins to learn the user's various characteristics but intensive monitoring insures that that data being evaluated is accurate. This computer automation is realized by improving the prior art through the use of rules applied to the available data, rather than a subjective determination by a doctor or therapist, and improving that application by reconsidering weights and establishing relationships between the various indications and sequences observed and recorded by the platform/application applied in the form of rules which may be limited to rules with certain common characteristics, i.e., a genus.

The embodiments as claimed are not a monopolization of the basic tools of scientific and technological work. Instead, they focus on a specific means or method that improves the relevant technology (for example, the combinations of indications evaluated and how they are weighted), not claiming just the result or effect. Such would be to claim, for example, “preventing a relapse,” which on its own is not claimed, but instead a specific set of values, process, and implementing a result which is a specific output alert or other physical quantity derived therefrom or in accordance therewith. The various embodiments invoke a series of steps that together are unique and effective in relapse prevention. Importantly, other techniques have been known for decades, but they fail in areas the present various embodiments excel because of those steps/rules.

While any claimed embodiment may be in part implemented in computer software processed by a general-purpose computer (e.g., on a smart phone), the process is different than those previously used in substance abuse recovery. The known processes for example, include subjective determinations rather than specific, or formulated evaluations of collected information (e.g., mobile device collected data), much of which itself has never been used or in combination as claimed. The smart phone here is employed to perform a distinct process to help automate that task and in interaction with the platform. It is the incorporation of that and/or similar distinct processes, not the use of the computer, which improves the addict's recovery process and prospects.

Further, the automation goes beyond merely “organizing [existing] information into a new form” or carrying out a fundamental economic practice. In general, the embodiments (and claims thereto) may use a combined order of specific rules that renders information into a specific format that is then used and applied to create desired results: a combination of automated machine responses (e.g., vibrating alarm), contact, and physical intervention. Which in no way preempts any known process or processes having a same result using different steps or rules.

The human condition of addiction and recovery is very complex. This complexity permits development of a large range of alternative rules-based processes for identifying and preventing, or at least forestalling an imminent relapse. The claims use limited rules in a process specifically designed to achieve an improved technological result in the addition recovery process, therefore, is not directed to an abstract idea.

Further, the application and its various embodiments may be applied in a general health and well-being use for users not associated with or having dependency issues. The user may utilize the app to help realize various emotional cycles and provide a toolkit to help deal with the ups and downs of daily life and activities which can be especially overwhelming in the modern world.

In one embodiment, a method is provided for automatically tracking/supporting healthy ongoing recovery, accountability, and relapse prevention in individuals who suffer from Alcohol Use Disorder (AUD) and Substance Use Disorder (SUD), and other related disorders. Monitoring emotional, physiological, Vital signs, and geo-spatial cues. Tracking habits/patterns/routine to monitor changes/anomalies that lead to relapse. Social media, Mobile analysis, geospatial data, contact tracing, facial recognition, biosensors, and daily routine. Geospatial functions enable geo-fencing outlining predetermined geographical areas that are high risk in addition to safe locations such as an AA meeting. Subsequently the platform via the application calculates if action is taken and refers/suggests immediate solutions in real time. Algorithmic computations identify relapse conditions of a user and automatically applies relapse prevention techniques without human oversight. Feedback from users questionnaire, Machine learning, and algorithms establish a baseline and create oversite over multiple data points identifying and acting on deviations. Prompting users to take action well in advance of a full relapse event. In most cases the user is not attuned enough to pick up on triggers. The platform, an evidence based, multidisciplinary decision maker supporting all modalities that address recovery. The platform identifies triggers in advance of any human contribution. Facilitating electronic guidance without human intervention unless the event is extreme and needs acute assistance to avoid full relapse and possible overdoes.

The method may include obtaining a first set of rules for weighting scores of indications of relapse according to timed events and a sequence of the events, obtaining scores for a set of indications via a platform and integrated applications connected personal mobile device maintained in close proximity to an AUD/SUD user, including a facial tension (or stress) score, a self-assessment score, a movement score, physiology score, vital score, biometric data, and geospatial data. Identifying, tracking, and reporting in advance of the people, places, and things that lead to relapse. Machine Learning (ML) and AI algorithms that systematically learn trigger events associated with the individual user. Sending electronic guidance and warnings for the individual to take action. Generating reports that help guide future disruptions to structured recovery. This is well in advance of a full episode.

In one embodiment, for example, if the user's vital signs elevate, coupled with an abnormal facial capture, biosensor data deviating from normal levels, application interaction has decreased, mobile analysis data is inconsistent, and you are in a predetermined dangerous geographical location and/or in proximity of a known individual.

These indicators may trigger for example, an event that leads to an action by the platform. Which in this use case for example, the platform would detect these abnormalities collected from the aforementioned end data points. This would trigger immediate automatic electronic interaction with the user. Electronic interaction that for example, may ask related questions to determine further weighting, to capture the ‘emotional state’ of the user. For example, suggest evidence-based actions be taken by the individual, guidance to the closest meeting or other related services. If no action is taken by the user, the platform, then performs cross check analysis on data captured, verifies data anomalies are reaching dangerous levels, and can begin to incrementally notify people within the sober network or clinical care team, or a predetermined combination thereof (and may include or exclusively be any of the other automated, robotics, or other non-human activity related actions discussed herein). If data anomalies in these or other related areas persistent, without correction, the platform can perform a full Intervention on the user's behalf. Informing all contacts or specified predetermined contacts within the user's sober network and/or clinical care team. Avoiding relapse and/or overdose/suicide.

In another embodiment, a user, for example could incrementally be deviating from their daily routine and/or patterns and not attending regular AA meetings. The platform via an application will interact with the user automatically, promoting action be taken in various forms. Offering incentives for reengaging in recovery, suggesting nearby meetings and/or services providing directions, suggest calling someone in their sober network. In addition, based on predetermined parameters, the platform can electronically correspond on the user's behalf. For example, receiving an inbound call from someone within your sober network and/or clinical care team, friend, therapist/phycologist, and/or relative. Such call may be an automated bot in a preferred voice tone. These types of non-emergent actions can be configured during the user onboarding process.

The use cases above outline the underpinnings of our complex algorithms in addition to our monitoring an evidence-based Standard deviation related to ongoing recovery, relapse prevention and intervention. Statistical Standard deviation, contains Upper Control Limit (UCL) and Lower Control Limit (LCL), where by you outline a standard deviation. Such standard deviations (and related control limits) may be provided for emotional state, Physical state (e.g., vitals, other sensor data, etc.), geographic location, and metaphysical components that can be measured and compared/contrasted with past events of the user and others. In one embodiment, the states may be subdivided into specific emotional conditions such as depression, happiness, attentiveness, nervousness, etc., each having its own data points for determining and each having its own standard deviation UCL and LCL.

The Platform and integrated applications use the aforementioned data, establishes a baseline of the user's data, runs machine learning and Artificial intelligence to determine and monitor, for example, emotional state of the user. An immediate action is taken by the platform when the thresholds on the LCL and the UCL are broken. Emotional escalation and depression that deviates from the predetermined standard deviation. Reporting and taking action on all data approaching the LCL and/or UCLs.

The method continues by determining an intermediate relapse score by weighting and combining the indication scores according to the first set of rules, obtaining a second set of rules comprising a historical weight of past relapse scores and transitional parameters associated with the user and/or similarly situated individuals under similar conditions wherein relapse scores and transitional parameters directly attributable to the user are weighted higher than scores and parameters of similarly situated individuals which themselves are ranked and weighted higher or lower depending on degree of similarity to the user, applying the second set of rules to the intermediate relapse score to determine a final relapse score; and preparing a graphic reflective of the final relapse score and relapse score information output (which may be provided on for the user's device, therapists screen, for example) or used for further analysis and/or as a part in electronically prompting and guiding the user to take healthy safe action. In the event no action is taken individuals within the user's sober network can be notified to make contact. In extreme relapse Danger, notifying entire care team and coordinated physical response or Intervention avoiding full relapse episode or overdoes. Such rules include, for example, UCL and LCL values for emotional state, Physical state (e.g., vitals, other sensor data, etc.), location, geophysical and metaphysical.

The coordinated physical response may comprise one or more responsive measures intended to physically address the user's condition, including, for example, at least one or electronically prompting the user to change his course, asking or telling the user to perform further actions, in extreme conditions prompting a call from their respective sober network of peers or clinical care team. Machine learning and algorithms calculate severity level, scores deviate to a critical level will initiating a physical intervention initiated by electronically notifying the users predetermined sober network or clinical care team. The graphic may, for example, comprise a caricature representation of the user that morphs from a pre-relapse relaxed state to a current relapse state along with scaled graphs, bar charts, or other indicia of physiological and/or psychological indications currently impacting the user. In the case event of a full-intervention, the physical response is an automated response triggering human interaction as an emergency mechanism to avoid full relapse or overdose, and may include various electronic interference (such as asking the user to complete a task) configured to delay the user in an effort to buy time for physical intervention to be performed.

The transitional parameters may be associated with at least one transition from a steady state relapse condition to a declined or declining relapse condition. The first set of rules and weightings may be related to current physical circumstances of the user and the second set of rules and weighting may be derived from past epoch user experiences and outcomes from physical experiences.

The user self-assessment may include, for example, questions that the user answers and rating the answers in comparison to previous self-assessments. A movement score may include an analysis to track the users movements over time assess the user's movements in comparison to previous similar situations. The physiological score may include, for example, current blood pressure and heart rate of the user and trends of the same over an immediately preceding period of time. A tension or stress score may be determined by taking images of the user, comparing the images to baseline images, and determine a score, wherein the score comprises a deviation from baseline of a plurality of lines on the user's face comprising percentage increase in depth and/or width of the lines or bay comparison to previous images and establishing a level of differences between the images.

In one embodiment, a medical device is provided for automatically monitoring relapse condition of an AUD/SUD user and automatically applying relapse prevention techniques without human oversight. This may be accomplished via a platform configured to obtain a first set of rules for weighting scores of indications of relapse according to timed events and a sequence of the events and obtain scores for a set of indications via a platform connected personal mobile device maintained in close proximity to a user diagnosed with Substance Use Disorder (SUD) and/or Alcohol Use Disorder (AUD), including a facial tension score, a self-assessment score, a movement score, and a physiology score. An intermediate relapse score may then be determined by weighting and combining the indication scores according to the first set of rules.

A second set of rules may be obtained that may include historical weight(s) of past relapse scores and transitional parameters associated with the user and/or similarly situated individuals under similar conditions. The relapse scores and transitional parameters directly attributable to the user may be weighted higher than scores and parameters of similarly situated individuals which themselves are ranked and weighted higher or lower depending on degree of similarity to the user (higher similarity=higher weightings). The second set of rules may then be applied to the intermediate relapse score to determine a final relapse score.

An application (e.g., on the users mobile device) may be configured to display a graphic representation of the final relapse score and relapse score information output for the user's review and a coordinated physical response. The coordinated physical response may be, for example, one or more responsive measures intended to physically address the user's condition, such as asking or telling the user to change his course, asking or telling the user to perform further tests, calling the user, and initiating a physical intervention.

The graphic may be, for example, a caricature representation of the user that morphs from a pre-relapse relaxed state to a current relapse state along with scaled graphs, bar charts, or other indicia of physiological and/or psychological indications currently impacting the user. In the case of a physical-intervention, the physical response may be in-part configured to delay the user in an effort to buy time for physical intervention to be performed. The transitional parameters may be, for example, associated with at least one transition from a steady state relapse condition to a declined or declining relapse condition. Instead of the morph (or the morph may include) a flipping back and forth between the caricature an image (e.g., mug shot) of the user.

If the various tasks or task is competed, additional tasks may be assigned to further delay the user (e.g., until an intervention arrives). For example, a phone operator may discuss new tasks to keep the user engaged until the intervention arrives, and/or the phone operator may engage in a multi-user gaming, or gambling operations with the user until the intervention arrives.

The first set of rules and weightings may be related to current physical circumstances of the user and the second set of rules and weighting are derived from past epoch user experiences and outcomes from physical experiences. The user self-assessment may include, for example, questions that the user answers and rating the answers in comparison to previous self-assessments. The movement score may be, for example, an analysis derived from tracking the user's movements over time assess the user's movements in comparison to previous similar situations. The physiological score may be, for example, current blood pressure and heart rate of the user and their trends over an immediately preceding period of time.

The stress/tension score may be determined by taking images of the user, comparing the images to baseline images, and determine a score, wherein the score comprises a deviation from baseline of a plurality of lines on the user's face comprising percentage increase in depth and/or width of the lines.

The application described may be an application or a remote process manages an intervention by contacting and verifying responders including a mix of professionals, volunteers, and stakeholders. The data collection comprises physiological data from a wearable device. Biometric data including, for example, the physiological data may include a gathering apparatus comprises a Galvanic Skin Response (GSR) detector. An AI module may be utilized to correlate GSR data to events and/or emotion cues to predict a current status of the user and invoke at least one of a query and interventional response if such prediction indicates a relapse event may occur or has occurred.

In one embodiment, a system of monitoring emotional health is provided including a smartphone configured to collect physiological and emotional data about a user, utilize the collected data to determine a user's likelihood of a relapse, and coordinate a response before, during, or shortly after the relapse. The physiological data may be derived, for example, from a fitness-like device comprising one of a Fitbit, heart rate monitor, blood pressure device, galvanic skin response detector, treadmill, Stairmaster, stationary bike, an electronic system at a gym, or like devices/systems.

An emotional query module configured to ask a user how they are feeling, and/or the user may be queried at one of regular time slots, periodic time slots, random time slots, upon events which may be associated with changes in emotional state, and events upon which the user has a history of emotional state changes, and wherein query responses combined with empirical data collected about the user's condition are together compared to previous similar conditions and emotional states to determine the relapse likelihood. The events may be, for example, combinations of more than one event or factor that affect emotional status.

A notification module may alert at least one of medical personnel, stakeholders, or family member if a relapse appears likely. The alert may include, for example, information about the user's current condition and location data of the user.

An emotional cue module may operate to query the user/individual for emotional status information including a snapshot or video comprising at least one of a selfie, facial shot, circumstances, or surroundings. The module may record interactions of the user with the emotional cue module, and, in the event of a deviation or other concerning circumstances, forwards the recorded interaction to a health professional. The module may record user/individual interactions with the app or system via a mobile device camera along with contemporaneous biometric data (forming a combination that may later be used to predict current condition of the user). An analysis of the photos or videos to establish or confirm an emotional state of the user/individual, such analysis including consideration of the contemporaneous biometric data including galvanic skin response may be performed.

Although the various embodiments have been described herein with reference mainly to dependencies such as alcohol and/or drug abuse any type of dependency may be addressed by the same and/or similar processes.

In describing the embodiments, and as illustrated in the drawings, specific terminology is employed for the sake of clarity. However, the various embodiments are not intended to be limited to the specific terminology so selected (or drawings provided), and it should be understood that the ordinarily skilled artisan may utilize similar, related, or even different terminology depending on the embodiment or selected topic therein to discuss, describe, or implement the same. Further, it should be understood that each specific element includes all technical equivalents which operate in a similar manner, as will be understood by the artisan. For example, when describing a mobile device (e.g., cell phone), any other equivalent device, such as a notebook, vehicle communication system (e.g., on-star), tablet, smart TV, or other device having an equivalent function or capability, whether or not listed herein, may be substituted therewith. Furthermore, the system and processes may be conveniently spread across a plurality of devices including various sensors that collect data used by the system and/o app. Still further, the inventor recognizes that newly developed technologies not now known may also be substituted for the described parts and still not depart from the scope of the present invention. All other described items, including, but not limited to processes, data collection, communications, protocols, responses, etc. should also be considered in light of any and all available equivalents.

Portions of the various embodiments may be conveniently implemented using a conventional general purpose or a specialized digital computer or microprocessor programmed according to the teachings of the present disclosure, as will be apparent to those skilled in the computer art.

Appropriate software coding can readily be prepared by skilled programmers based on the teachings of the present disclosure, as will be apparent to those skilled in the software art. The various embodiments or portions thereof, may also be implemented by the preparation of application specific integrated circuits or by interconnecting an appropriate network of conventional component circuits, as will be readily apparent to those skilled in the art based on the present disclosure. Such circuits may include, for example, biometric sensors (e.g., wearables, patches, implants, touch sensitive devices, etc.), chemical detectors, reflex tests/observations, IR scanners (e.g., Flir camera images), etc. Any or all of which may be combined with data points or other information as described above to analyze current situations or provide information for later analysis and/or comparisons.

The various embodiments include a computer program product which is a storage medium (media) having instructions stored thereon/in which can be used to control, or cause, a computer to perform any of the processes of the embodiments. The storage medium can include, but is not limited to, any type of disk including floppy disks, mini disks (MD's), optical discs, DVD, HD-DVD, Blue-ray, CD-ROMS, CD or DVD RW+/−, micro-drive, cloud storage, and magneto-optical disks, ROMs, RAMs, EPROMs, EEPROMs, DRAMs, VRAMs, flash memory devices (including flash cards, memory sticks), magnetic or optical cards, SIM cards, MEMS, nanosystems (including molecular memory ICs), RAID devices, remote data storage/archive/warehousing, or any type of media or device suitable for storing instructions and/or data.

Stored on any one of the computer readable medium (media), the embodiments may include software for controlling both the hardware of the general purpose/specialized computer or microprocessor, and for enabling the computer or microprocessor to interact with a human user or other mechanism utilizing the results of any embodiment or variations/equivalents thereof. Such software may include, but is not limited to, device drivers, operating systems, and user applications. Ultimately, such computer readable media further includes software for performing any embodiment as described above and equivalents thereof.

Included in the programming (software) of the general/specialized computer or microprocessor are software modules for implementing the teachings of the various embodiments, including, but not limited to, identification of environment and other circumstances, retrieval of stored information and comparison to statistical data and past circumstances in support of any of the above, and the display, storage, or communication of results according to the processes as described herein and equivalent processes whether or not described herein.

The embodiments may suitably comprise, consist of, or consist essentially of, any of element (the various parts or features of the embodiments and their equivalents as described herein). Further, the embodiments illustratively disclosed herein may be practiced in the absence of any element, whether or not specifically disclosed herein. Obviously, numerous modifications and variations of each embodiment are possible in light of the above teachings. It is therefore to be understood that within the scope of any claims, the invention, or any embodiment thereof, may be practiced otherwise than as specifically described herein.

What is claimed is:

1. A medical device automatically monitoring relapse condition of an AUD/SUD user and automatically applying relapse prevention techniques without human oversight, comprising:
a platform configured to,
obtain a first set of rules for weighting scores of indications of relapse according to timed events and a sequence of the events,
obtain scores for a set of indications via a platform connected personal mobile device maintained in close proximity to a user diagnosed with Substance Use Disorder (SUD) and/or Alcohol Use Disorder (AUD), including a facial stress tension score, a self-assessment score, a movement score, and a physiology score,
determine an intermediate relapse score by weighting and combining the indication scores according to the first set of rules,
obtain a second set of rules comprising a historical weight of past relapse scores and transitional parameters associated with the user and/or similarly situated individuals under similar conditions wherein relapse scores and transitional parameters directly attributable to the user are weighted higher than scores and parameters of similarly situated individuals which themselves are ranked and weighted higher or lower depending on degree of similarity to the user, and
apply the second set of rules to the intermediate relapse score to determine a final relapse score; and
an application configured to display a graphic representation of the final relapse score and relapse score information output for the user's review and a coordinated physical response;
wherein, the coordinated physical response comprises one or more responsive measures intended to physically address the user's condition, including at least one of,
asking or telling the user to change his course,
asking or telling the user to perform further tests,
calling the user, and
initiating a physical intervention.

2. The medical device according to claim 1, wherein the graphic comprises a caricature representation of the user that morphs from a pre-relapse relaxed state to a current relapse state along with scaled graphs, bar charts, or other indicia of physiological and/or psychological indications currently impacting the user.

3. The medical device according to claim 1, wherein, in the case of a physical-intervention, the physical response is in part configured to delay the user in an effort to buy time for physical intervention to be performed.

4. The method according to claim 1, wherein the transitional parameters are associated with at least one transition from a steady state relapse condition to a declined or declining relapse condition.

5. The method according to claim 1, wherein the first set of rules and weightings are related to current physical circumstances of the user and the second set of rules and weighting are derived from past epoch user experiences and outcomes from physical experiences.

6. The method according to claim 1, wherein the user self-assessment includes questions that the user answers and rating the answers in comparison to previous self-assessments.

7. The method according to claim 1, wherein the movement score comprises an analysis to track the users movements over time assess the user's movements in comparison to previous similar situations.

8. The method according to claim 1, wherein the physiological score comprising current blood pressure and heart rate of the user and their trends over an immediately preceding period of time.

9. The method according to claim 1, wherein the stress score is determined by taking images of the user, comparing the images to baseline images, and determine a score, wherein the score comprises a deviation from baseline of a plurality of lines on the user's face comprising percentage increase in depth and/or width of the lines.

10. A method for automatically treating a relapse condition of a recovering user and providing a smart phone based coordinated physical response comprising:
obtaining a first set of rules that together define output stream of data representing a likelihood of relapse as a function of time place, mood related to users past actions and past actions of similarly situated individuals;
obtaining a data file of current user conditions comprising a plurality of sub-conditions of conditions comprising at least one of events, moods, locations related to a user's current situation and recent activities;
generating an intermediate stream of output data weighted based on a similarity of current and recent events to the output stream of data and further weighted to the extent the output stream of data represents the user's past data by evaluating said plurality of sub-sequences against said first set of rules;
generating a final determination of likelihood of relapse based on the weighted intermediate stream of output data and selecting a presentation format and actions for the user's smart phone and auxiliary resources to address the likely relapse; and
applying said final determination in the selected presentation format on the user's smart phone comprising at least one of controlling the smart phone such that the user must address further actions before using the smart phone in any capacity other than to address the potential relapse, providing a pop display informing the user of conditions currently being experienced and having the user acknowledge the same, providing options for the user to proceed in a particular direction or on a particular route; and
applying the auxiliary resources by sending alert messages to trained team members with a data set of the user's condition, location including an interactive map showing a current location and course of the user, and coordinating communication with the user.

11. The method according to claim 10, wherein the first set of rules comprises a upper and lower limits around a standard deviation of the user's location and emotional state.

12. The method according to claim 10, wherein coordinating communication with the user comprises coordinating communication with the user while enroute to intervene.

13. The method according to claim 10, wherein actions for the user's smart phone include at least one of disabling data communication, sounding an alert, requiring a self-assessment, and directing a user to change course.

14. The method according to claim 10, wherein the standard deviations include a combination of subjective evaluation(s) of the user's condition and empirical evaluation(s) of the user's condition.

15. The method according to claim 10, wherein the standard deviations (and related control limits) are provided for emotional state, vitals (BP, Heartrate, etc.), and geographic location.

16. The method according to claim 10, wherein the standard deviations include Such standard deviations metaphysical components that can be measured and compared/contrasted with past events of the user and others.

17. A system comprising:
a platform configured to maintain data stores of end-point data and derive standard deviations for a plurality of user end-points, wherein the end-point data stores comprises emotional state, physical state, geographic location, and metaphysical components that can be measured and compared/contrasted with past events of the user and others;
a mobile device application configured to retrieve data related to each of the end-points;
a rules-based evaluation configured to compare a user's current condition related to each of the end-points with respect to its corresponding standard deviation and determine a relapse condition of the user; and
a display of the mobile device configured to display the user's current condition in a manner that makes the user realize his/her current condition in both good times and in bad and provide one of a suggestion as to a diversion the user should engage and/or an order to divert depending on the severity or risk that the condition imposes for the user.

18. The system according to claim 17, wherein the platform is further configured to weight the user's condition based on a combination of end-points.

19. The system according to claim 17, wherein the rules-based evaluation comprises evaluating at least one data point in combination with a location of the user where values of the data point at the user's location over time provides a baseline and standard deviation from which to evaluate a current value of the data point.

20. The system according to claim 17, wherein the application is in communication with biometric data gathering apparatus linked to the individual.
